(19) United States
(12) Patent Application Publication
(10) Pub. No.: US 2026/0124146 A1
(43) Pub. Date: May 7, 2026

(54) MODIFIED RELEASE TOLCAPONE FORMULATIONS

(71) Applicant: Corino Therapeutics, Inc., New York, NY (US)

(72) Inventors: Michael Roberts, Charlotte, NC (US); Harish Pimplaskar, Pineville, NC (US); Paul Glidden, San Diego, CA (US)

(21) Appl. No.: 19/119,980

(22) PCT Filed: Oct. 6, 2023
(86) PCT No.: PCT/US2023/076194

§ 371 Date: Apr. 10, 2025

Related U.S. Application Data
(60) Provisional application No. 63/414,655, filed on Oct. 10, 2022.

(51) Int. Cl. A61K 9/24; A61K 9/20; A61K 31/121

(52) U.S. Cl. CPC A61K 9/209; A61K 9/2027; A61K 9/2054; A61K 31/121

(57) ABSTRACT

Modified release tablet dosage forms comprising tolcapone are disclosed. The tablet dosage forms provide a pulsed, pH dependent release profile of tolcapone to both the gastric space and the small intestine. Methods of treating or preventing a disease selected from transthyretin amyloidosis (ATTR), Parkinson's Disease and obsessive compulsive disorder using said dosage forms are provided.

### CROSS-REFERENCE TO RELATED APPLICATION

This application claims priority to U.S. Provisional Application No. 63/414,655, filed Oct. 10, 2022, the contents of which are incorporated by reference herein.

### FIELD OF THE INVENTION

The present disclosure relates to modified release tablets of tolcapone.

### BACKGROUND

Transthyretin (ATTR) amyloidosis includes a group of amyloid diseases specifically associated with transthyretin (TTR) protein. TTR is a 127 amino acid 55 KD homo-tetrameric produced primarily in the liver and secreted into the plasma. Dissociation of the TTR-tetramer at the T4-binding interface generates monomers that misfold and aggregate to form amyloid fibrils. These fibrils, together with unstable fibril precursors, produce cell death and tissue damage.

ATTR is characterized by deposition of misfolded protein in one or more organ systems (including the peripheral and autonomic nervous systems, the heart, the brain and the eyes). The age at which symptoms begin to develop varies widely ranging between 20 to 70 years old. ATTR is progressive, and some variants can have a fatal outcome within a few years of presentation. Treatment options include supportive and symptomatic care that may slow or stop progressive decline in functional state but do not alter the pathological process. Liver transplant can be performed in selected patients but is limited by organ supply, requires lifelong immunosuppression, and may be complicated by progressive heart and nerve amyloid deposition. Importantly, liver transplant does not alter the natural course of central nervous system amyloid disease. Life expectancy is generally between 5 and 15 years following diagnosis.

TTR dissociation can be the result of a genetic mutation (hereditary ATTR), aging (wt ATTR) or both. There are more than 120 known amyloidogenic mutations in TTR with diverse clinical manifestations, including hATTR-polyneuropathy (familial amyloid polyneuropathy (FAP)) and hATTR-cardiomyopathy (familial amyloid cardiomyopathy (FAC)). Often, hereditary ATTR (hATTR) results in a mix of progressive neurological and cardiological impairment. hATTR-leptomeningeal is an under recognized, progressive and fatal disease caused by accumulation in the brain of variant transthyretin (TTR) expressed by the choroid plexus, causing central nervous system (CNS) dysfunction.

Tolcapone is one of several small molecules that can stabilize the tetrameric structure of TTR, reducing or preventing dissociation (Sant′Anna R. et al. Nature Communications volume 7, Article number: 10787 (2016). Tolcapone is FDA-approved for treatment of Parkinson's disease and crosses the blood-brain barrier.

A recent proof-of-concept study of orally dosing hATTR-leptomeningeal patients three times daily with 100 mg tolcapone over 14 days and then three times daily with 200 mg tolcapone over an additional 14 days demonstrated that tolcapone is a potent TTR stabilizer in both plasma and cerebral spinal fluid (CSF). Tolcapone normalized TTR concentration in plasma with an overall mean increase in plasma TTR tetramer concentration of 55%. Significant concentrations of drug penetrated into the CSF decreasing monomeric TTR concentration by a mean of 48% as measured under semi-denaturing conditions. (Berk J., Kaku, M., Alosco, M., Lazzari, V., Brueckner, C., Doros, G., Glidden, P., Roberts, M., Tolcapone Levels and TTR

Stabilization in Cerebrospinal Fluid of Patients with Leptomeningeal Amyloid TTR Mutations, XVII International Symposium on Amyloidosis, Abstract PW020, September 2020).

However, tolcapone is eliminated fairly quickly, with an elimination half-life of 1.6 to 3.4 hours (Keating G M, Lyseng-Williamson K A. Tolcapone: a review of its use in the management of Parkinson's disease. CNS Drugs. 2005; 19 (2): 165-84). Immediate release dosage forms are therefore not suitable for treatment ATTR as 4-6 dosings a day would be required to ensure consistent TTR stabilization (e.g., at nighttime). Patient compliance with such dosing regimens would be problematic, thereby reducing efficacy.

Initial attempts to formulate modified release tolcapone formulations utilized off-the-shelf technology using wet granulation with HPMC binder. A 300 mg tablet was developed containing (i) an intragranular portion containing tolcapone (37.50 wt %), HPMC E50 (22.0 wt %), HPMC K100LV (10.0 wt %), dicalcium phosphate anhydrous (24.75 wt %) and (ii) an extragranular component containing Aerosil 200 (0.50 wt %) and magnesium stearate (1.0 wt %). A Phase I study in healthy subjects unexpectedly indicated low bioavailability and minimal extended release (FIG. 1).

Further investigations demonstrated that exposure to low pHs found in the stomach changed the nature of the tablet such that it became rubberized and gelatinous and resisted dissolution after transition to the higher pHs found in the small intestine. It was also found that tolcapone itself has a strong pH dependent solubility, with lower pHs associated with low tolcapone solubility and a physical change in appearance from crystalline to longer, hair-like structures.

It is hypothesized that the rearranged, hair-like tolcapone form is less soluble, which leads to slow dissolution and poor drug release in the low pH environs of the stomach.

Therefore, there remains a need for modified release oral dosage forms of tolcapone that can be administered twice daily and provide sustained TTR stabilization.

### SUMMARY

Disclosed herein are tablet dosage forms of tolcapone that provide a pulsed release profile useful for treating conditions such as ATTR, and can be dosed twice daily for consistent TTR stabilization.

A tablet disclosed herein comprises:
(i.) a tablet core comprising a first portion of tolcapone, and optionally, at least one binder, filler, glidant and/or lubricant;
(ii.) an optional film coating layer surrounding the tablet core and comprising at least one cellulosic polymer;
(iii.) an enteric coating layer surrounding the core or film coating layer and comprising an acrylic acid and methacrylic acid copolymer, and optionally at least one additive;
(iv.) an immediate release layer surrounding the enteric coating layer and comprising a second portion of tolcapone and at least one cellulosic polymer; and
(v.) an optional top coat surrounding the immediate release layer.

Also provided are methods of treating a disease in a patient, comprising orally administering to a patient in need thereof the tablets disclosed herein. In particular, the tablets disclosed herein can be used to treat any disease or condition for which tolcapone is indicated including ATTR and Parkinson's Disease.

In exemplary embodiments, the patient receives no more than two doses daily, each dose comprising from about 100 mg to about 600 mg tolcapone, more preferably from about 100 mg to about 300 mg tolcapone. Each dose can be one tablet or multiple tablets disclosed herein.

The ATTR is selected from hereditary ATTR (hATTR), hATTR-polyneuropathy (hATTR-PN), hATTR-cardiomyopathy (hATTR-CM), ATTR-cardiomyopathy (ATTR-CM), hATTR-Leptomeningeal (hATTR-Lepto) and mixed phenotypes thereof.

### BRIEF DESCRIPTION OF THE DRAWINGS

FIG. 1 is a graph showing the release profile of (i) Tasmar® in a fasted state (circles) and (ii) a prototype modified release tablet containing tolcapone according to Example 1 in the fasted state (diamonds).

FIG. 2 is an illustration of a tablet according to the present invention.

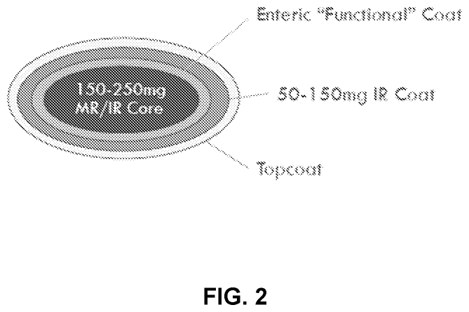

FIG. 3 is a graph showing the plasma concentrations of tolcapone over time in Part 1 of the PK study (Example 4).

FIG. 4 is a graph showing the mean fraction of initial (FOI) and mean plasma concentration over time for Prototype 1 (Example 5).

FIG. 5 is a flow-chart illustrating preparation of tablets described herein (Example 3).

### DEFINITIONS

“AUC” refers to the area under a curve on which time is plotted on the X-axis and concentration of a substance (e.g., tolcapone) in blood or blood plasma is plotted on the Y-axis over a particular period of time (e.g., time zero to 24 hours). AUC is commonly expressed in units of ng·hr/ml.

“Average Cmax” refers to the average of two or more individual Cmax values determined across a group of multiple subjects. For example, if multiple subjects are dosed as described herein they can each have a different individual Cmax value. The calculated mean of these different Cmax values is the “Average Cmax” for the group.

“Average Cmin” refers to the average of two or more individual Cmax values determined across a group of multiple subjects. For example, if multiple subjects are dosed as described herein they can each have a different individual Cmin value. The calculated mean of these different Cmin values is the “Average Cmin” for the group.

“Disease” refers to a disease, disorder, condition, or symptom of any of the foregoing.

“Gastrointestinal tract” or “GI tract” refers to the digestive tract, a muscle-membrane tube that is about 30 feet long and extends from the mouth to the anus. As used herein, the term “upper gastrointestinal tract” refers to the buccal cavity, pharynx, esophagus, stomach and small intestine. As used herein, the term “lower gastrointestinal tract” refers to the large intestine and rectum.

“Immediate release” refers to formulations or dosage forms that rapidly dissolve in vitro and in vivo and are intended to be completely dissolved and absorbed in the stomach or upper gastrointestinal tract. Immediate release formulations can release at least 90% of the active ingredient or precursor thereof within about 15 minutes, within about 30 minutes, within about one hour, or within about two hours of administering an immediate release dosage form.

“Patient” or “subject” refers to a mammal, for example, a human.

“Modified release” (used interchangeably with “sustained release” or “Delayed Release” or Controlled Release”) refers to release of a drug from a dosage form in which the drug release occurs over a period of time. Modified release can mean that release of the drug from the dosage form is extended for longer than it would be in an immediate-release dosage form, i.e., at least over several hours. In some embodiments, in vivo release of tolcapone occurs over a period of about 6 hours, about 7 hours, about 8 hours, about 9 hours, about 10 hours, about 11 hours, or about 12 hours.

“Small intestine” refers to the portion of the gastrointestinal tract consisting of the duodenum, the jejunum and the ileum, i.e., the portion of the intestinal tract immediately adjacent to the duodenal sphincter of the upper base and proximate to the large intestine.

“Treating” or “treatment” of any disease refers to reversing, alleviating, arresting, or ameliorating a disease or at least one of the clinical symptoms of a disease, reducing the risk of acquiring at least one of the clinical symptoms of a disease, inhibiting the progress of a disease or at least one of the clinical symptoms of the disease or reducing the risk of developing at least one of the clinical symptoms of a disease. “Treating” or “treatment” also refers to inhibiting the disease, either physically, (e.g., stabilization of a discernible symptom), physiologically, (e.g., stabilization of a physical parameter), or both, and to inhibiting at least one physical parameter that may or may not be discernible to the patient. In certain embodiments, “treating” or “treatment” refers to protecting against or delaying the onset of at least one or more symptoms of a disease in a patient.

### DETAILED DESCRIPTION

### I. Tablets

In preferred embodiments, the tablets of the present invention provide a pulsed, pH dependent release profile of tolcapone to both the gastric space and, after a lag period, the small intestine, when administered to a subject. Such a release profile requires the appropriate selection of multiple coating layers.

The immediate release layer of the tablets described herein is soluble/dissolves within the gastric space at stomach pH (pH 1-3). During this phase, the portion of tolcapone present in the immediate release layer is released. The enteric coating is not soluble at gastric pH and therefore protects the core (and the portion of tolcapone contained therein) during this phase. There is then a lag time until the remainder of the tablet enters the small intestine. The enteric coating and core is soluble/dissolves in the neutral or slightly alkaline pH of the small intestine (pH 6 in the duodenum; pH 6-7.5 in jejunum and ileum), thereby providing release of the portion of tolcapone within the core and a second pulse of active to the subject.

### A. Tablet Core

Tablets disclosed herein comprise a tablet core containing tolcapone. Tolcapone in the tablet core is present in an amount from about 10% to about 95% by weight of the total tablet weight, such as, for example, from about 10 wt % to about 90 wt %, from about 10 wt % to about 80 wt %, from about 10 wt % to about 70 wt %, from about 10 wt % to about 60 wt %, from about 10 wt % to about 50 wt %, from about 10 wt % to about 40 wt %, from about 10 wt % to about 30 wt %, from about 10 wt % to about 20 wt %, from about 20 wt % to about 95 wt %, from about 20 wt % to about 90 wt %, from about 20 wt % to about 80 wt %, from about 20 wt % to about 70 wt %, from about 20 wt % to about 60 wt %, from about 20 wt % to about 50 wt %, from about 20 wt % to about 40 wt %, from about 20 wt % to about 30 wt %, from about 30 wt % to about 95 wt %, from about 30 wt % to about 90 wt %, from about 30 wt % to about 80 wt %, from about 30 wt % to about 70 wt %, from about 30 wt % to about 60 wt %, from about 30 wt % to about 50 wt %, from about 30 wt % to about 40 wt %, from about 40 wt % to about 95 wt %, from about 40 wt % to about 90 wt %, from about 40 wt % to about 80 wt %, from about 40 wt % to about 70 wt %, from about 40 wt % to about 60 wt %, from about 40 wt % to about 50 wt %, from about 50 wt % to about 95 wt %, from about 50 wt % to about 90 wt %, from about 50 wt % to about 80 wt %, from about 50 wt % to about 70 wt %, from about 50 wt % to about 60 wt %, from about 60 wt % to about 95 wt %, from about 60 wt % to about 90 wt %, from about 60 wt % to about 80 wt %, from about 60 wt % to about 70 wt %, from about 70 wt % to about 95 wt %, from about 70 wt % to about 90 wt %, from about 70 wt % to about 80 wt %, from about 80 wt % to about 95 wt %, from about 80 wt % to about 90 wt %, or from about 90 wt % to about 95 wt %.

In a particular embodiment, tolcapone in the tablet core is present in an amount from about 20% to about 30% by weight of the total tablet weight.

The tablet core comprises tolcapone in an amount from about 10% to about 95% by weight of the total tablet core weight, such as, for example, from about 10 wt % to about 90 wt %, from about 10 wt % to about 80 wt %, from about 10 wt % to about 70 wt %, from about 10 wt % to about 60 wt %, from about 10 wt % to about 50 wt %, from about 10 wt % to about 40 wt %, from about 10 wt % to about 30 wt %, from about 10 wt % to about 20 wt %, from about 20 wt % to about 95 wt %, from about 20 wt % to about 90 wt %, from about 20 wt % to about 80 wt %, from about 20 wt % to about 70 wt %, from about 20 wt % to about 60 wt %, from about 20 wt % to about 50 wt %, from about 20 wt % to about 40 wt %, from about 20 wt % to about 30 wt %, from about 30 wt % to about 95 wt %, from about 30 wt % to about 90 wt %, from about 30 wt % to about 80 wt %, from about 30 wt % to about 70 wt %, from about 30 wt % to about 60 wt %, from about 30 wt % to about 50 wt %, from about 30 wt % to about 40 wt %, from about 40 wt % to about 95 wt %, from about 40 wt % to about 90 wt %, from about 40 wt % to about 80 wt %, from about 40 wt % to about 70 wt %, from about 40 wt % to about 60 wt %, from about 40 wt % to about 50 wt %, from about 50 wt % to about 95 wt %, from about 50 wt % to about 90 wt %, from about 50 wt % to about 80 wt %, from about 50 wt % to about 70 wt %, from about 50 wt % to about 60 wt %, from about 60 wt % to about 95 wt %, from about 60 wt % to about 90 wt %, from about 60 wt % to about 80 wt %, from about 60 wt % to about 70 wt %, from about 70 wt % to about 95 wt %, from about 70 wt % to about 90 wt %, from about 70 wt % to about 80 wt %, from about 80 wt % to about 95 wt %, from about 80 wt % to about 90 wt %, or from about 90 wt % to about 95 wt %.

In a particular embodiment, the tablet core comprises tolcapone in an amount from about 40% to about 50% by weight of the tablet core weight.

The tolcapone can be micronized or non-micronized tolcapone. In preferred embodiments, the tolcapone is micronized.

Compressed tablet cores containing tolcapone can be made using well-known techniques such as those described in Remington: The Science and Practice of Pharmacy, 21^{st} Edition, University of the Sciences in Philadelphia Ed. (2005). Such tablet cores can contain one or more known tableting excipients such as binders, fillers, disintegrants, glidants, lubricants, surfactants, plasticizers, anti-adherents, buffers, disintegrants, wetting agents, emulsifying agents, thickening agents, coloring agents, sustained release agents, or combinations of any of the foregoing.

Binders may be included in the tablet core to hold the components of the core together. Exemplary binders include, alginic acid and salts thereof; cellulose derivatives such as carboxymethylcellulose, methylcellulose (e.g., Methocel®), hydroxypropyl methylcellulose, hydroxyethylcellulose, hydroxypropyl cellulose (e.g., Klucel®), ethylcellulose (e.g., Ethocel®), and microcrystalline cellulose (e.g., Avicel®); microcrystalline dextrose; amylose; magnesium aluminum silicate; polysaccharide acids; bentonites; gelatin; polyvinylpyrrolidone/vinyl acetate copolymer; crospovidone; povidone; starch; pregelatinized starch; tragacanth, dextrin, a sugar, such as sucrose (e.g., Dipac®), glucose, dextrose, molasses, mannitol, sorbitol, xylitol (e.g., Xylitab®), and lactose; a natural or synthetic gum such as acacia, tragacanth, ghatti gum, mucilage of isapol husks, polyvinylpyrrolidone (e.g., Polyvidone® CL, Kollidon® CL, Polyplasdone® XL-10), larch arabogalactan, Veegum®, polyethylene glycol, waxes, sodium alginate, and combinations of any of the foregoing.

In particular embodiments, the binder is hydroxypropyl methylcellulose. Many different grades of hydroxypropyl methylcellulose exist depending on, e.g., the molecular weight thereof, the degree of etherification, viscosity etc. Hydroxypropyl methyl celluloses include “E”, “F” and “K” chemistry products available from Dow Chemical Company under the tradename METHOCEL®. Each hydroxypropyl methylcellulose is available in a variety of viscosities. An exemplary, commercially available hydroxypropyl cellulose is obtainable from e.g. JRS Pharma under the trade name VIVAPHARM® HPMC-E.

In a particular embodiment, the hydroxypropyl methylcellulose has a viscosity (mPa·s) of 3.0-5.0 as measured in an aqueous solution containing 2% by weight of dry HPMC at 20° C. In another particular embodiment, the binder is HPMC-E.

The at least one binder in the core is present in an amount from about 1% to about 10% by weight of the total tablet weight, such as, for example, from about 2 wt % to about 6 wt %.

Fillers may be added to increase the bulk to make dosage forms. Examples of fillers include, for example, dibasic calcium phosphate, dibasic calcium phosphate dihydrate, calcium sulfate, dicalcium phosphate, tricalcium phosphate, lactose, cellulose including microcrystalline cellulose, mannitol, sodium chloride, dry starch, pregelatinized starch, compressible sugar, mannitol, and combinations thereof. Fillers may be water insoluble, water soluble, or combinations thereof. Examples of useful water insoluble fillers include starch, dibasic calcium phosphate dihydrate, calcium sulfate, dicalcium phosphate, tricalcium phosphate, powdered cellulose, microcrystalline cellulose, and combinations thereof. Examples of water-soluble fillers include water soluble sugars and sugar alcohols, such as lactose, glucose, fructose, sucrose, mannose, dextrose, galactose, the corresponding sugar alcohols and other sugar alcohols, such as mannitol, sorbitol, xylitol, and combinations thereof. In particular embodiments, the at least one filler is microcrystalline cellulose.

The at least one filler in the core is present in an amount from about 5% to about 50% by weight of the total tablet weight, such as, for example, from about 5 wt % to about 40 wt %, from about 5 wt % to about 30 wt %, from about 5 wt % to about 20 wt %, from about 5 wt % to about 15 wt % or from about 5 wt % to about 10 wt. In a particular embodiment, the at least one filler in the core is present in an amount from about 20% to about 30% by weight of the total tablet weight.

Glidants or anti-adherents may be included in the tablet core to reduce sticking effects during processing, film formation, and/or drying. Examples of useful glidants include talc, glycerol monostearate, colloidal silicon dioxide, precipitated silicon dioxide, fumed silicon dioxide, and combinations thereof. In particular embodiments, the glidant is fumed silicon dioxide.

The at least one glidant is present in an amount from about 0.1% to about 1% by weight of the total tablet weight, such as, for example, from about 0.1 wt % to about 0.5 wt %.

Lubricants and anti-static agents may be included to aid in processing. Examples of lubricants include calcium stearate, glyceryl monostearate, magnesium stearate, mineral oil, polyethylene glycol, sodium stearyl fumarate, sodium lauryl sulfate, stearic acid, talc, vegetable oil, zinc stearate, and combinations thereof. In particular embodiments, the lubricant is magnesium stearate.

The at least one lubricant in the core is present in an amount from about 0.1% to about 1% by weight of the total tablet weight, such as, for example, from about 0.1 wt % to about 0.5 wt %.

Disintegrants may be included in the tablet core to cause a tablet core to break apart, for example, by expansion of a disintegrants when exposed to water. Examples of useful disintegrants include water swellable substances such as croscarmellose sodium, sodium starch glycolate, cross-linked polyvinyl pyrrolidone, and combinations thereof.

In various embodiments, the core contains an immediate release formulation of tolcapone.

In alternative embodiments, the tablet core may also be a sustained release formulation. Such tablets would provide an extended release throughout the small and large intestines. Examples of polymer materials for effecting sustained release include, but are not limited to, cellulosic polymers such as hydroxypropyl cellulose, hydroxyethyl cellulose, hydroxymethyl cellulose, hydroxypropyl methylcellulose, hydroxypropyl methylcellulose acetate succinate, hydroxypropyl methylcellulose phthalate, methylcellulose, ethyl cellulose, cellulose acetate, cellulose acetate phthalate, cellulose acetate trimellitate, and carboxymethylcellulose sodium. Combinations of any of the foregoing polymers may also be used.

### B. Film Coating Layer

A film coating layer optionally surrounds the tablet core. The film coating can fill-in any imperfections of the tablet core and provide a smooth surface for application of the enteric coating.

In one embodiment, the film coating layer comprises at least one cellulosic polymer. Exemplary cellulosic polymers include, but are not limited to, methylcellulose, ethylcellulose, propylcellulose, butylcellulose, cellulose acetate, cellulose propionate, cellulose butyrate, cellulose acetate butyrate, cellulose acetate propionate, methyl cellulose, methyl cellulose acetate, methyl cellulose propionate, methyl cellulose butyrate, ethyl cellulose acetate, ethyl cellulose propionate, ethyl cellulose butyrate, hydroxymethyl cellulose, hydroxyethyl cellulose, hydroxyethyl methyl cellulose, hydroxypropyl cellulose, hydroxybutyl cellulose, hydroxyethyl cellulose acetate, and hydroxyethyl ethyl cellulose, low-substituted hydroxypropyl cellulose, hydroxypropyl methylcellulose, hydroxypropyl methylcellulose acetate, hydroxypropyl methylcellulose propionate, hydroxypropyl methylcellulose butyrate, and corresponding salts and esters. In particular embodiments, the film coating comprises hydroxypropyl methylcellulose.

In a particular embodiment, the film coating cellulosic polymer is a type of Opadry® and/or OPAGLOS® 2 film-coating system manufactured by Colorcon of West Point, Pa. Opadry® | and/or Opadry® II are preferred.

The film coating is present in an amount from about 0.1% to about 10% by weight of the total tablet weight, such as, for example, from about 1 wt % to about 5 wt % or about 1 wt % to about 3 wt %.

### C. Enteric Coating Layer

An enteric coating layer surrounds the core or the optional film coating layer. Enteric polymer coatings operate on the principle of pH dependent solubility: insoluble in the low pH conditions of the stomach but soluble in the near neutral pH environment of the proximal small intestine.

The enteric coating begins to dissolve in an aqueous solution at pHs greater than 5.0, 5.5, 6.0 or 6.5. The enteric coating layer protects the contents of the tablet core, preventing degradation of the core and/or tolcapone release from the core in acidic environs of the stomach and prevents tablet gelation during the pH transition from the stomach to the small intestine.

The enteric coating layer comprises at least one of the following polymers: shellac, gelatin, methacrylic acid copolymer type C NF, cellulose butyrate phthalate, cellulose hydrogen phthalate, cellulose proprionate phthalate, polyvinyl acetate phthalate (PVAP), cellulose acetate phthalate (CAP), cellulose acetate trimellitate (CAT), hydroxypropyl methylcellulose phthalate, hydroxy propyl methylcellulose acetate, dioxypropyl methylcellulose succinate, carboxymethyl ethylcellulose (CMEC), hydroxy propyl methylcellulose acetate succinate (PMCAS), and acrylic acid polymers and copolymers, typically formed from methyl acrylate, ethyl acrylate, methyl methacrylate and/or ethyl methacrylate with copolymers of acrylic and methacrylic acid esters, acrylic acid and methacrylic acid copolymers, methyl methacrylate copolymers, ethoxyethyl methacrylates, cyanoethyl methacrylate, poly(acrylic acid), poly(methacrylic acid), methacrylic acid alkylamide copolymer, poly(methyl methacrylate), polymethacrylate, poly(methyl methacrylate) copolymers, polyacrylamide, aminoalkyl methacrylate copolymer, poly(methacrylic acid anhydride), glycidyl methacrylate copolymers, ammonioalkyl methacrylate copolymers, and methacrylic resins commercially available under the tradename Eudragit® including Eudragit®, L, Eudragit® S, Eudragit®E, Eudragit® RL, and Eudragit® RS. In particular embodiments, the polymer is a copolymer based on methacrylic acid and ethyl acrylate, e.g., Eudragit® L or Eudragit® L30 D55 from Evonik Industries AG.

The enteric coating polymer is present in an amount from about 0.5% to about 10% by weight of the total tablet weight, such as, for example, from about 0.5 wt % to about 5 wt %, from about 0.5% to about 3.0 wt %, from about 0.5 wt % to about 2.0% wt, or from about 1.0 wt % to about 2.0 wt %. In a particular embodiment, the enteric coating is present in an amount from about 1.0% to about 7% by weight of the total tablet weight.

The enteric coating layer can further comprise at least one additive, e.g., anti-tacking agents, plasticizers and stabilizers. Exemplary anti-tacking additives include PlasACRYL®s, 20% aqueous emulsions of glycerol monostearate as an anti-tacking agent and triethyl citrate as a plasticizer and stabilizer. PlasACRYL® HTP20 was designed for Eudragit® L30 D55 formulations. PlasACRYL® T20 was designed for Eudragit® FS 30 D formulations. In particular embodiments, the at least one additive is PlasACRYL® HTP20.

The at least one enteric coating layer additive is present in an amount from about 0.1% to about 1.0% by weight of the total tablet weight, such as, for example, from about 0.1 wt % to about 4.0 wt %, from about 0.1 wt % to about 3.0 wt %, from about 0.1 wt % to about 2.0 wt %, from about 0.1 wt % to about 1.0 wt %.

### D. Immediate Release Layer

An immediate release layer surrounds the enteric coating. The immediate release layer comprises tolcapone and provides immediate release and absorption of tolcapone following dissolution of the enteric coating in the small intestine.

Tolcapone in the immediate release layer is present in an amount from about 10% to about 95% by weight of the total tablet weight, such as, for example, from about 10 wt % to about 90 wt %, from about 10 wt % to about 80 wt %, from about 10 wt % to about 70 wt %, from about 10 wt % to about 60 wt %, from about 10 wt % to about 50 wt %, from about 10 wt % to about 40 wt %, from about 10 wt % to about 30 wt %, from about 10 wt % to about 20 wt %, from about 20 wt % to about 95 wt %, from about 20 wt % to about 90 wt %, from about 20 wt % to about 80 wt %, from about 20 wt % to about 70 wt %, from about 20 wt % to about 60 wt %, from about 20 wt % to about 50 wt %, from about 20 wt % to about 40 wt %, from about 20 wt % to about 30 wt %, from about 30 wt % to about 95 wt %, from about 30 wt % to about 90 wt %, from about 30 wt % to about 80 wt %, from about 30 wt % to about 70 wt %, from about 30 wt % to about 60 wt %, from about 30 wt % to about 50 wt %, from about 30 wt % to about 40 wt %, from about 40 wt % to about 95 wt %, from about 40 wt % to about 90 wt %, from about 40 wt % to about 80 wt %, from about 40 wt % to about 70 wt %, from about 40 wt % to about 60 wt %, from about 40 wt % to about 50 wt %, from about 50 wt % to about 95 wt %, from about 50 wt % to about 90 wt %, from about 50 wt % to about 80 wt %, from about 50 wt % to about 70 wt %, from about 50 wt % to about 60 wt %, from about 60 wt % to about 95 wt %, from about 60 wt % to about 90 wt %, from about 60 wt % to about 80 wt %, from about 60 wt % to about 70 wt %, from about 70 wt % to about 95 wt %, from about 70 wt % to about 90 wt %, from about 70 wt % to about 80 wt %, from about 80 wt % to about 95 wt %, from about 80 wt % to about 90 wt %, or from about 90 wt % to about 95 wt %.

In certain embodiments, tolcapone in the immediate release layer is present in an amount from about 20% to about 30% by weight of the total tablet weight.

Tolcapone is suspended or solubilized using at least one cellulosic polymer, which is then film coated over the enteric coating layer. The at least one cellulosic polymer can be the same or different as the at least one cellulosic polymer used in the optional film coating layer. In certain embodiments, the at least one cellulosic polymer comprises hydroxypropyl methylcellulose. In a particular embodiment, the cellulosic polymer is a type of Opadry® and/or OPAGLOS® 2 film-coating system manufactured by Colorcon of West Point, Pa. Opadry® | and/or Opadry® II are preferred.

The at least one cellulosic polymer in the immediate release layer is present in an amount from about 5% to about 20% by weight of the total tablet weight, such as, for example, from about 5 wt % to about 15 wt %, from about 5 wt % to about 10 wt % or from about 10 wt % to about 15 wt %.

The tablet comprises from about 100 mg to about 1,000 mg tolcapone, which is divided between the core (first portion) and the immediate release layer (second portion), such, as, for example, from about 100 mg to about 900 mg, from about 100 mg to about 800 mg, from about 100 mg to about 700 mg, from about 100 mg to about 600 mg, from about 100 mg to about 500 mg, from about 100 mg to about 400 mg, from about 100 mg to about 300 mg and from about 100 mg to about 200 mg.

The amount of tolcapone in the core (first portion) can vary from about 50 mg to about 900 mg, such as, for example, from about 50 mg to about 500 mg, from about 50 mg to about 300 mg, from about 50 mg to about 200 mg or from about 50 mg to about 100 mg.

The amount of tolcapone in the immediate release layer (second portion) can vary from about 50 mg to about 900 mg, such as, for example, from about 50 mg to about 500 mg, from about 50 mg to about 300 mg, from about 50 mg to about 200 mg or from about 50 mg to about 100 mg.

The weight ratio of the first portion of tolcapone to the second portion of tolcapone can vary from about 1:5 to about 5:1, such as, for example, from about 1:4 to about 4:1, from about 1:3 to about 3:1, from about 1:2 to about 2:1 or about 1:1. In a particular embodiment, the weight ratio is about 1:1.

The total tablet weight can vary from 100 mg to about 1,000 mg, such as, for example, from about 100 mg to about 300 mg, from about 100 mg to about 500 mg, from about 200 mg to about 300 mg, from about 200 mg to about 500 mg, from about 300 mg to about 500 mg, and from about 400 mg to about 500 mg.

### E. Top Coat

The tablet optionally includes a top coat which can be spray-dried over the immediate release layer. Exemplary top coating materials include at least one cellulosic polymer (e.g., hydroxypropyl methylcellulose, Opadry®), HPC, Eudragit® RL, Eudragit® E100, Eudragit® E 12.5, Eudragit® E PO, Eudragit® NE and combinations thereof. In a particular embodiment, the top coat comprises hydroxypropyl methylcellulose.

The top coat can be present in an amount from about 0.1% to about 10% by weight of the total tablet weight, such as, for example, from about 0.1 wt % to about 7.0 wt %, from about 0.1 wt % to about 5.0 wt %, from about 0.1 wt % to about 3.0 wt %, from about 0.1 wt % to about 2.0 wt % or about 0.1 wt % to about 1.0 wt %. In a particular embodiment, the top coat is present in an amount from about 0.1% to about 3.0% by weight of the total tablet by weight.

In an exemplary embodiment, a tablet of the present invention comprises:
(i.) a tablet core comprising a first portion of tolcapone, and optionally, at least one binder, filler, glidant and/or lubricant;
(ii.) an optional film coating layer surrounding the tablet core and comprising at least one cellulosic polymer;
(iii.) an enteric coating layer surrounding the core or optional film coating layer and comprising an acrylic acid and methacrylic acid copolymer, and optionally at least one additive;
(iv.) an immediate release layer surrounding the enteric coating layer and comprising a second portion of tolcapone and at least one cellulosic polymer; and
(v.) an optional top coat surrounding the immediate release layer.

In a more particular embodiment, a tablet of the present invention comprises:
(i.) a tablet core comprising a first portion of tolcapone, and optionally:
   a. at least one binder, wherein the binder, if present, is hydroxypropyl methyl cellulose;
   b. at least one filler, wherein the filler, if present, is microcrystalline cellulose;
   c. at least one glidant, wherein the glidant, if present, is fumed silicon dioxide; and/or
   d. at least one lubricant, wherein the lubricant, if present, is magnesium stearate;
(ii.) an optional film coating layer surrounding the tablet core and comprising at least one cellulosic polymer;
(iii.) an enteric coating layer surrounding the core or optional film coating layer and comprising an acrylic acid and methacrylic acid copolymer, and optionally at least one additive comprising an aqueous suspension of an anti-tacking agent, a plasticizer and stabilizer;
(iv.) an immediate release layer surrounding the enteric coating layer and comprising a second portion of tolcapone and at least one cellulosic polymer; and
(v.) an optional top coat surrounding the immediate release layer and comprising at least one cellulosic polymer.

In a more particular embodiment, a tablet of the present invention comprises, by weight:
(i.) a tablet core comprising a first portion of tolcapone, wherein the first portion of tolcapone is present in an amount from about 10 wt % to about 95 wt %, and optionally:
   a. at least one binder in an amount from about 1 wt % to about 10 wt %,
   b. at least one filler in an amount from about 5 wt % to about 50 wt %,
   c. at least one glidant in an amount from about 0.1 wt % to about 1 wt %, and/or at least one lubricant in an amount from about 0.1 wt % to about 1 wt %;
(ii.) an optional first film coating layer surrounding the tablet core and comprising at least one cellulosic polymer, wherein the at least one cellulosic polymer is present in an amount from about 0.1 wt % to about 10 wt %;
(iii.) an enteric coating layer surrounding the core or optional film coating layer and comprising an acrylic acid and methacrylic acid copolymer, wherein the copolymer is present in an amount from about 0.5 wt % to about 10 wt %, and optionally at least one additive in an amount from about 0.1 wt % to about 1.0 wt %;
(iv.) an immediate release layer surrounding the enteric coating layer and comprising a second portion of tolcapone and at least one cellulosic polymer, wherein the second portion of tolcapone is present in an amount from about 10 wt % to about 95 wt %; and the cellulosic polymer is present in an amount from about 5 wt % to about 20 wt % and
(v.) an optional top coat surrounding the immediate release layer and comprising at least one cellulosic polymer, wherein the top coat is present in an amount from about 0.1 wt % to about 10 wt %.

In a more particular embodiment, a tablet of the present invention comprises, by weight:
(i.) a tablet core comprising a first portion of tolcapone, wherein the first portion of tolcapone is present in an amount from about 20 wt % to about 30 wt %, and optionally:
   a. at least one binder in an amount from about 2 wt % to about 10 wt %,
   b. at least one filler in an amount from about 20 wt % to about 30 wt %,
   c. at least one glidant in an amount from about 0.1 wt % to about 0.5 wt %, and/or at least one lubricant in an amount from about 0.1 wt % to about 0.5 wt %;
(ii.) an optional film coating layer surrounding the tablet core and comprising at least one cellulosic polymer, wherein the at least one cellulosic polymer is present in an amount from about 1 wt % to about 3 wt %;
(iii.) an enteric coating layer surrounding the core or optional film coating layer and comprising an acrylic acid and methacrylic acid copolymer, wherein the copolymer is present in an amount from about 1.0 wt % to about 4.0 wt %, and optionally at least one additive in an amount from about 0.1 wt % to about 1.0 wt %;
(iv.) an immediate release layer surrounding the enteric coating layer and comprising a second portion of tolcapone and at least one cellulosic polymer, wherein the second portion of tolcapone is present in an amount from about 20 wt % to about 30 wt % and the at least one cellulosic polymer is present in an amount from about 5 wt % to about 15 wt %; and
(v.) an optional top coat surrounding the immediate release layer and comprising at least one cellulosic polymer, wherein the top coat is present in an amount from about 0.1 wt % to about 3.0 wt %.

In another exemplary embodiment, a tablet of the present invention comprises:
(i.) a tablet core comprising a first portion of tolcapone and at least one sustained release polymer, and optionally, at least one binder, filler, glidant and/or lubricant;
(ii.) an optional film coating layer surrounding the tablet core and comprising at least one cellulosic polymer;
(iii.) an enteric coating layer surrounding the core or film coating layer and comprising acrylic acid an methacrylic acid copolymer, and optionally at least one additive;
(iv.) an immediate release layer surrounding the enteric coating layer and comprising a second portion of tolcapone at least one cellulosic polymer; and
(v.) an optional top coat surrounding the immediate release layer and comprising at least one cellulosic polymer.

Tablets of the present invention can provide an immediate release of tolcapone within the gastric space (from the immediate release layer), and then subsequent release (immediate or sustained) of tolcapone from the tablet core in the small intestine. The structure of the tablet prevents gelation and associated release problems observed with previous tolcapone formulations.

The in vitro dissolution rate describes how the amount of tolcapone released changes over time when subjected to an in vitro dissolution test. A higher/faster in vitro dissolution rate means that a larger amount of tolcapone is release over a certain period of time, and a lower/slower in vitro dissolution rate means that a smaller amount of tolcapone is released over the same period of time when subjected to the same in vitro dissolution testing conditions.

The in vitro dissolution of the tablet can be measured by placing a tablet in 1000 ml of the dissolution medium (pH 6.8 phosphate buffer containing 0.5% SLS) and stirring at 75 rpm and 37±0.5° C. Samples are then taken at regular intervals (e.g., every 15 minutes for up to 24 hours) and tolcapone concentration is measured using HPLC.

In exemplary embodiments, the tablets disclosed herein release at least 95% of the tolcapone when placed in the dissolution medium within about 1 hour, about 2 hours, about 3 hours, about 4 hours or about 5 hours. In a preferred embodiment, the tablet releases at least 95% of the tolcapone when placed in the dissolution medium within about 1-5 hours, about 1-4 hours, about 2-5 hours, about 3-5 hours, about 2-4 hours or about 3-4 hours.

### II. Therapeutic Uses

The tablets disclosed herein may be administered to a patient suffering from any disease including a disorder, condition, or symptom for which tolcapone is known or hereafter discovered to be therapeutically effective. Indications for which tolcapone is also expected to be effective include ATTR, Parkinson's Disease, schizophrenia, polycystic kidney disease and obsessive compulsive disorder.

Methods of treating a disease in a patient provided by the present disclosure comprise administering to a patient in need of such treatment the tablets disclosed herein. The tablets disclosed herein may provide therapeutic or prophylactic plasma and/or blood concentrations of tolcapone following administration to a patient.

The tablets disclosed herein may be administered in an amount and using a dosing schedule as appropriate for treatment of a particular disease. For example, daily doses of tolcapone may range from about 0.01 mg/kg to about 50 mg/kg, such as, for example, from about 1 mg/kg to about 50 mg/kg, from about 1 mg/kg to about 40 mg/kg, from about 1 mg/kg to about 30 mg/kg, from about 1 mg/kg to about 20 mg/kg or from about 1 mg/kg to about 10 mg/kg.

In particular embodiments, daily doses of tolcapone range from about 2 mg/kg to about 10 mg/kg, from about 3 mg/kg to about 10 mg/kg, from about 4 mg/kg to about 10 mg/kg, from about 5 mg/kg to about 10 mg/kg, from about 6 mg/kg to about 10 mg/kg, from about 7 mg/kg to about 10 mg/kg, from about 8 mg/kg to about 10 mg/kg or from about 9 mg/kg to about 10 mg/kg.

In certain embodiments, the tolcapone may be administered at a dose of from about 1 mg to about 1 g per day, from about 100 mg to about 600 mg, from about 300 mg to about 600 mg per day, or from about 100 mg to about 300 mg per day. An appropriate dose of tolcapone may be determined based on several factors, including, for example, the body weight and/or condition of the patient being treated, the severity of the disease being treated, the incidence and/or severity of side effects, the manner of administration, and the judgment of the prescribing physician. Appropriate dose ranges may be determined by methods known to those skilled in the art.

A dose may be administered in a single tablet or in multiple tablets. When multiple tablets are used the amount of tolcapone contained within each dosage form may be the same or different.

In certain embodiments, a dose is administered to a patient twice daily. In preferred embodiments, not more than two doses are administered to a patient daily.

The dose can be administered in a fed or fasted state. In certain embodiments, the dose is administered in a fasted state. In certain other embodiments, the dose is administered in a fed state.

In certain embodiments, a therapeutically effective dose of tolcapone may provide therapeutic benefit without causing substantial toxicity including adverse side effects.

Administration of the tablets disclosed herein provides an average maximum blood plasma concentration (Average Cmax) of tolcapone of about 1,000 ng/ml to about 10,000 ng/ml, such as, for example, from about 1,000 ng/ml to about 8,000 ng/ml, 1,000 ng/ml to about 5,000 ng/ml, 1,000 ng/ml to about 3,000 ng/ml, 5,000 ng/ml to about 10,000 ng/ml, 5,000 ng/ml to about 8,000 ng/mL, about 7,000 ng/ml to about 10,000 ng/ml or about 8,000 ng/ml to about 10,000 ng/mL.

Administration of the tablets disclosed herein provides a minimum average blood plasma concentration (Average Cmin) of tolcapone of no less than 200 ng/ml 12 hours after dosing, no less than 500 ng/ml 12 hours after dosing or no less than 800 ng/ml 12 hours after dosing.

In certain embodiments, administration provides a minimum average blood plasma concentration (Average Cmin) of from about 200 ng/ml to about 800 ng/ml 12 hours after dosing.

Administration of the tablets disclosed herein provides an area under a concentration of tolcapone in blood plasma versus time curve (AUC_{0-inf}) of at least about 3,200 ng·hr/ml, such as, for example, from about 3,500 ng·hr/ml to about 4,000 ng·hr/ml. In some embodiments, the tolcapone AUC_{0-inf} is from about 3,500 ng·hr/ml to about 4,000 ng·hr/ml when dosed in the fasted state. In other embodiments, the tolcapone AUC_{0-inf} is from about 3,200 ng·hr/ml to about 3,500 ng·hr/ml when dosed in the fed state.

In certain embodiments, administration of a tablet of the present invention to a patient releases no more than 60% of the tolcapone contained therein within 2 hours following administration, such as, for example, no more than 50% of the tolcapone within 2 hours following administration, no more than 40% of the tolcapone within 2 hours following administration or no more than 30% tolcapone within 2 hours following administration.

In certain embodiments, administration of a tablet of the present invention to a patient releases no less than 90% of the tolcapone contained therein within 8 hours following administration, or no less than 95% tolcapone within 8 hours following administration.

In certain embodiments, administration of a tablet of the present invention to a patient release no more than 60% of the tolcapone contained therein within 2 hours and no less than 90% of the tolcapone contained therein within 9 hours.

The tablets of the present invention provide a first pulse of tolcapone upon dissolution of the immediate release layer, which occurs from 0-4 hours following administration. A second pulse of tolcapone is released upon dissolution of the enteric coating when the remainder of the tablet reaches the small intestine, around 5-6 hours following administration. In preferred embodiments, release is complete after about 8 hours.

### A. Transthyretin Amyloidosis (ATTR)

In a particular embodiment, a method for treating transthyretin amyloidosis (ATTR) in a patient comprises administering at least one tablet disclosed herein comprising an effective amount of tolcapone to said patient.

In one embodiment, the ATTR is hereditary ATTR, i.e., ATTR primarily resulting from one or more pathogenic mutations in the TTR gene. Generally, hATTR impacts the nerves, heart, kidneys, eyes, and brain. The rate of cardiac versus neurological involvement depends on the underlying TTR mutation.

In a particular embodiment, the one or more pathologic mutations in the TTR gene are selected from single nucleotide substitution, deletion or duplication. The particular mutation may be homogenous or heterogenous. In certain embodiments, the ATTR caused by one or more pathogenic mutations. In one embodiment, the one or more pathogenic mutations impacts the C-D loop of the TTR protein.

In a particular embodiment, the one or more pathogenic mutation is selected from the group consisting of Gly6Ser, Cys10Arg, Leu12Pro, Leu12Val, Met13Ile, Asp18Asn, Asp18Gly, Asp18Glu, Ala19Asp, Val20Ile, Arg21Gln, Ser23Asn, Pro24Ser, Ala25Ser, Ala25Thr, Val28Met, Val28Ser, Val30Leu, Val30Met, Val30Ala, Val30Gly, Val30Leu, Val32Ala, Val32Gly, Phe33Ile, Phe33Leu, Phe33Val, Phe33Cys, Arg34Gly, Arg34Thr, Lys35Asn, Lys35Thr, Ala36Asp, Ala36Pro, Asp38Ala, Asp38Val, Thr40Asn, Trp41Leu, Glu42Gly, Glu42Asp, Phe44Tyr, Phe44Ser, Phe44Leu, Ala45Ser, Ala45Thr, Ala45Asp, Ala45Gly, Gly47Arg, Gly47Glu, Gly47Val, Thr49Ala, Thr49Pro, Thr49Ile, Thr49Ser, Ser50Arg, Ser50Ile, Glu51Gly, Glu51_Ser52dup, Ser52Pro, Gly53Arg, Gly53Glu, Gly53Ala, Glu54Leu, Glu54Lys, Glu54Gly, Glu54Asp, Glu54Gln, Leu55Gln, Leu55Arg, Leu55Pro, His56Arg, Gly57Arg, Leu58Arg, Leu58His, Thr59Arg, Thr59Lys, Thr60Ala, Thr60Ile, Glu61Lys, Glu61Gly, Glu61Ala, Glu62Lys, Phe64Ile, Phe64Leu, Phe64Ser, Phe64Val, Gly67Arg, Gly67Glu, Ile68Leu, Tyr69His, Tyr69Ile, Lys70Asn, Val71Ala, Glu72Gly, Ile73Val, Asp74His, Ser77Phe, Ser77Tyr, Tyr78Phe, Ala81Thr, Ala81Val, Gly83Arg, Ile84Asn, Ile84Ser, Ile84Thr, His88Arg, Glu89Gln, Glu89Lys, His90Asn, His90Asp, Ala91Ser, Gln92Lys, Val93Met, Val94Ala, Ala97Ser, Ala97Gly, Gly101Ser, Pro102Arg, Arg103Ser, Arg104Cys, Arg104His, Ile107Val, Ile107Phe, Ile107Met, Ala109Ser, Ala109Thr, Ala109Val, Leu111Met, Ser112Ile, Pro113Thr, Tyr114Cys, Tyr114His, Tyr114Ser, Tyr116Ser, Thr116Met, Ala120Ser, Val122del, Val 122Ile, Val122Ala and Pro125Ser.

In a particular embodiment, the pathogenic mutation underlying the hereditary ATTR is Val30Met (i.e., substitution of valine for methionine in position 30 of the transthyretin protein). Sousa A, et al. Am J Med Genet. 1995; 60:512-521).

In certain embodiments, the subject treated according to the present invention has asymptomatic hATTR resulting from Val30Met, early-onset hATTR resulting from Val30Met or late-onset hATTR resulting from Val30Met.

In a particular embodiment, the ATTR is hATTR-polyneuropathy (hATTR-PN). hATTR-PN is a generally caused by a genetic mutation (i.e., a point mutation) in the transthyretin gene, with V30M being the most common. The age of onset of hATTR-PN can vary widely, from early-stage (>about 40 years old) to late-stage (>about 50 years old). hATTR-PN is clinically heterogeneous, on the mutation and the subject's geographic origin. hATTR-PN usually presents as a length-dependent sensory polyneuropathy with autonomic disturbances. Symptoms of peripheral neuropathy include, e.g., tingling, pins and needles in the feet and hands; weakness and pain in the arms and legs; loss of sensation (numbness); and loss of thermal sensibility in the feet. Symptoms of automatic neuropathy include, e.g., postural hypotension; disturbed bowel function, nausea, vomiting; urinary retention; impotence; and reduced sweating. Average survival is about 10 years after symptoms present. As the patients age, central manifestations begin to develop.

In another particular embodiment, the ATTR is hATTR-cardiomyopathy (hATTR-CM). hATTR-CM presents clinically as heart disease (restrictive cardiomyopathy) and sometimes carpal tunnel syndrome, with the latter often occurring years before the former. In a particular embodiment, the mutant TTR is V122I. Symptoms of hATTR-CM include, e.g., chest pain (angina), shortness of breath; palpitations and abnormal heart rhythms; ankle swelling (edema) fatigue; nausea; weight loss; and dizziness.

In another embodiment, the ATTR is caused primarily by wild-type (wt) transthyretin and more particularly, is clinically manifest as ATTR-cardiomyopathy (ATTR-CM). Symptoms include slowly progressive energy decline, exercise intolerance, weight loss and gastro-intestinal (GI) complaints, as well as left and right sided congestive heart failure (CHF), with normal systolic function and clear diastolic dysfunction, arrythmias.

In another embodiment, the ATTR is hATTR-Leptomeningeal (hATTR-Lepto). hATTR-Lepto is caused primarily by an ultra-rare genetic mutation in TTR that causes predominantly central pathology through accumulation of amyloid in the meninges of the brain.

The period of treatment can vary. In one embodiment, treatment is carried out for at least three months, such as, for example, four months, five months, six months, seven months, eight months, nine months, ten months, eleven months or twelve months. In a particular embodiment, the subject treated according to the method described herein exhibits a slowing of disease progression or an improvement in disease as measured by the subject's Neuropathic Impairment Score (NIS) or modified Neuropathic Impairment Score (mNIS).

In a particular embodiment, the subject treated according to the method described herein exhibits a slowing of disease progression or an improvement in disease as measured by the subject's report of Transient Focal Neurological Episodes (TFNEs).

In a particular embodiment, the subject treated according to the method described herein exhibits a slowing of disease progression or an improvement in disease as measured by the subject's Norfolk Quality of Life-Diabetic Neuropathy (QOL-DN) score. The Norfolk QOL-DN patient-reported questionnaire that comprises domains for physical functioning/large-fiber neuropathy, symptoms, activities of daily life, small-fiber neuropathy, and autonomic neuropathy.

In a particular embodiment, the subject treated according to the method described herein exhibits a slowing of disease progression or an improvement in disease as measured by the subject's Kansas City Cardiomyopathy Questionnaire (KCCQ).

In another embodiment, the subject treated according to the methods described herein exhibits an absence of clinically significant changes in most clinical, biochemical, electrocardiographic echocardiographic parameters, consistent with delayed cardiac disease progression.

In another particular embodiment, the subject treated according to the method described herein exhibits an improvement in the subject's modified body mass index (mBMI) compared to a reference subject treated with conventional (immediate release) tolcapone. In a particular embodiment, the subject's mBMI is improved by an amount between about 1% and about 20%, more particularly, between about 1 and about 10%, more particularly about 1%, about 2%, about 3%, about 4%, about 5%, about 6%, about 7%, about 8%, about 9% or about 10%.

In another particular embodiment, the subject treated according to the method described herein exhibits an improvement, compared to conventional (immediate release) tolcapone, in the subject's 6-minute walk test” or “6MWT”—a test that measures the distance that a patient can quickly walk on a flat, hard surface in a period of 6 minutes (the 6MWD). In a particular embodiment, the subject's 6 minute walk test is improved by an amount between about 1% and about 20%, more particularly, between about 1 and about 10%, more particularly about 1%, about 2%, about 3%, about 4%, about 5%, about 6%, about 7%, about 8%, about 9% or about 10%.

In a particular embodiment, the subject treated according to the method described herein exhibits a slowing of disease progression or an improvement in disease as measured by a reduction in hospitalizations or mortality.

In still another particular embodiment, the subject treated according to the methods disclosed herein exhibits TTR tetramer stabilization of at least about 20%, at least about 30%, at least about 40%, at least about 50% or at least about 60% for at least 10 hours following administration, such as, for example, from about 10 hours to about 14 hours or from about 10 hours to about 12 hours. By stabilizing the tetrameric form, the formation of TTR amyloid is reduced or prevented.

TTR stabilization can be measured using an immunoturbidity assay as described in Gamez, J., et al., “Transthyretin stabilization activity of the catechol-O-methyltranslferase inhibitor tolcapone (SOM0226) in hereditary ATTR amyloidosis patients and asymptomatic carriers: proof-of-concept study, Amyloid 2019.

In another particular embodiment, the subject treated according to the method disclosed herein exhibits one or more of the following characteristics following administration, compared to a reference subject treated with conventional tolcapone: (i) an increase in the level of tetrameric TTR (ii) a decrease the level of monomeric TTR and/or (iii) an increase the ratio of tetrameric to monomeric TTR.

### B. Parkinson's Disease

In one embodiment, a method for treating Parkinson's Disease in a patient comprises administering at least one tablet disclosed herein comprising an effective amount of tolcapone to said patient.

Parkinson's disease is a slowly progressive degenerative disorder of the nervous system characterized by tremor when muscles are at rest (resting tremor), slowness of voluntary movements, and increased muscle tone (rigidity). In Parkinson's disease, nerve cells in the basal ganglia, e.g., substantia nigra, degenerate, and thereby reduce the production of dopamine and the number of connections between nerve cells in the basal ganglia. As a result, the basal ganglia are unable to smooth muscle movements and coordinate changes in posture as normal, leading to tremor, incoordination, and slowed, reduced movement (bradykinesia) (Blandini, et al., Mol. Neurobiol. 1996, 12, 73-94).

The efficacy of the tablets disclosed herein for treating Parkinson's disease may be assessed using animal and human models of Parkinson's disease and in clinical studies.

### C. Obsessive Compulsive Disorder

Grant, R. et al recently reported that tolcapone may improve obsessive compulsive disorder (Grant, R., et al., Tolcapone in obsessive-compulsive disorder: a randomized double-blind placebo-controlled crossover trial; Int Clin Psychopharmacol. 2021 Sep. 1; 36(5): 225-229).

In one embodiment, a method for treating obsessive compulsive disorder (OCD) in a patient comprises administering at least one tablet disclosed herein to said patient.

In obsessive-compulsive disorder, the primary symptom is recurrent obsessions (i.e., recurrent and intrusive thoughts, images or urges that cause marked anxiety) and/or compulsions (i.e., repetitive behaviors or mental acts that are performed to reduce the anxiety generated by one's obsessions) of sufficient severity to cause distress, be time consuming or to interfere significantly with a person's normal routine or lifestyle. Anxiety is an associated feature of this disorder. An affected person may, for example, show a phobic avoidance of situations that involve the cause of the obsession. Typical obsessions concern contamination, doubting (including self-doubt) and disturbing sexual or religious thoughts. Typical compulsions include washing, checking, ordering, and counting.

A pervasive pattern of preoccupation with orderliness, perfectionism, and mental interpersonal control, at the expense of flexibility, openness, and efficiency, beginning by early adulthood and present in a variety of contexts, as indicated by four (or more) of the following:
1. Is preoccupied with details, rules, lists, order, organization, or schedules to the extent that the major point of the activity is lost.
2. Shows perfectionism that interferes with task completion.
3. Is excessively devoted to work and productivity to the exclusion of leisure activities and friendships (not accounted for by obvious economic necessity).
4. Is overconscientious, scrupulous, and inflexible about matters of morality, ethics or values.
5. Is unable to discard worn-out or worthless objects even when they have no sentimental value.
6. Is reluctant to delegate tasks or to work with others unless they submit to exactly his or her way of doing things.
7. Adopts a miserly spending style towards both self and others; money is viewed as something to be hoarded for future catastrophes.
8. Shows rigidity and stubbornness.

Thus, obsessive compulsive disorder can be characterized by at least 4, 5, 6, 7 or all 8 of these characteristics.

In one embodiment, the method comprises administering at least one of the present invention and an additional therapeutic agent. Exemplary additional therapeutic agents include, but are not limited to, selective serotonin reuptake inhibitors (SSRI) (e.g., paroxetine, sertraline, fluoxetine, escitalopram and fluvoxamine), tricyclic antidepressants (clomipramine), a benzodiazepine and atypical antipsychotics (e.g., olanzapine, quetiapine, and risperidone). The tablet of the present invention and the additional therapeutic agent can be administered together or sequentially.

The present methods can result in a decrease in one or more behaviors associated with obsessive compulsive disorder.

### EXAMPLES

The following examples illustrate various aspects of the disclosure. It will be apparent to those skilled in the art that many modifications, both to materials and methods, may be practiced without departing from the scope of the disclosure.

### Example 1

300 mg tolcapone modified release tablets according to Table 1 were prepared:

TABLE 1

| | 300 mg Tolcapone MR Tablet | | |
|---|---|---|---|
| Formulation | % w/w | mg/ tablet | Qty per 200 g |

Intragranular

| | | | |
|---|---|---|---|
| Tolcapone | 37.50 | 300.00 | 75.00 |
| HPMC E50 (fixed level) | 20.00 | 176.00 | 44.00 |
| HPMC E50 | 2.00 | | |
| (for dose compensation) | | | |
| HPMC K100LV | 10.00 | 80.00 | 20.00 |
| Dicalcium Phosphate Anhydrous | 24.75 | 198.00 | 49.50 |
| Lactose monohydrate | 4.25 | 34.00 | 8.50 |
| Subtotal | 98.50 | — | 197.00 |

Extragranular

| | | | |
|---|---|---|---|
| Aerosil 200 | 0.50 | 4.00 | 1.00 |
| Magnesium stearate | 1.00 | 8.00 | 2.00 |
| Total | 100.00 | 800.00 | 200.00 |

The dissolution profile of the tablet was determined under the following conditions:

| | |
|---|---|
| Apparatus: | USP Apparatus II (Paddles) |
| Medium: | 1000 mL dissolution medium |
| | pH 6.8 phosphate buffer containing 0.5% SLS |
| Paddle speed: | 75 rpm (250 rpm infinity) |
| Temperature: | 37 ± 0.5° C. |
| Sampling time points: | 1, 2 and 2.5 hours |
| Sampling type: | Automatic with 10 μm full flow filters |
| Sampling volume: | 1.5 mL |

Tolcapone concentration was measured using the following HPLC conditions:
HPLC column: Kromasil, C18, 4×250 mm, 100 A, 5 μm
Column Temperature: 40° C.
Sample temperature: Ambient
UV Detection: UV 271 nm
Flow rate: 1.0 mL/minute
Injection volume: 5.0 μL
Mobile Phase: 25:40:35 Acetonitrile: Methanol: Phosphate Buffer, 5 mM, pH 2.0
Needle wash: 100% methanol
Run time: 15 minutes

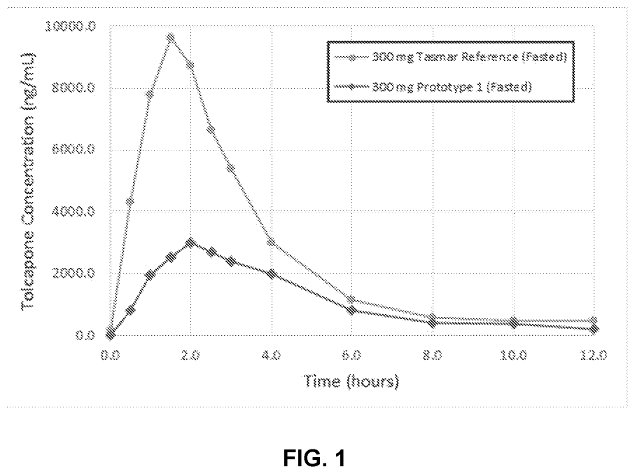

The dissolution profile was promising and indicated 80% drug release was achieved between 12-13 hours, which could have allowed for the possibility of once-a-day dosing had the pharmacokinetics been promising. However, a Phase 1 study in fasted and fed subjects showed significant lack of absorption of tolcapone (FIG. 1). It was hypothesized that poor bioavailability was at least partially due to retention of tolcapone in the formulation. Another concern was the possibility of regional absorption preventing extended release formulations from being a proper delivery system.

### Example 2

375±10 mg of micronized tolcapone was added to 150 mL of each buffer for solubility assessment, giving 2.5 mg of tolcapone/mL. Two aliquots of each sample were taken at 1 and 2 hour time points. One aliquot was used for Assay and Related Substances analysis by reverse phase HPLC and the other aliquot was used to obtain microscopic images using 5.5× magnification, polarised and non-polarised light. In addition, representative photograph of each n=1 sample at 1 hour time point were taken for information. pH of each sample was measured at 2 hour time point.

### Visual Observations

At pH 2.0, appearance of solution in both replicates went from clear and colorless (before the addition of tolcapone) to heavy cloudy and bright yellow within first 5 minutes (mins) (after the addition of tolcapone). No further change in appearance were observed.

At pH 4.5, appearance of solution in both replicates went from clear and colorless (before the addition of tolcapone) to heavy cloudy and yellow within first 5 mins (after the addition of tolcapone). No further changes in appearance were observed.

At pH 5.5, appearance of solution in both replicates went from clear and colorless (before the addition of tolcapone) to heavy cloudy and light orange within first 5 mins (after the addition of tolcapone). No further changes in appearance were observed.

At pH 6.8, appearance of solution in both replicates went from clear and colorless (before the addition of tolcapone) to clear and dark red within first 5 mins (after addition of tolcapone). No visible solids were observed. Both solutions later became opaque by 45 mins mark and significant amount of precipitation was observed by 1 hour mark. No further changes in appearance were observed.

At pH 7.4, appearance of solution in both replicates went from clear and colorless (before the addition of tolcapone) to heavy cloudy and orange within first 5 mins (after the addition of tolcapone). No further changes in appearance were observed.

The tolcapone solubility determined by HPLC is provided in Table 2.

TABLE 2

| | 1 Hour | | 2 Hour | |
|---|---|---|---|---|
| | Assay | Mean Assay | Assay | Mean Assay |
| Sample Name | (mg/mL) | (mg/mL) | (mg/mL) | (mg/mL) |
| pH 2.0 N = 1 | 0.023 | 0.020 | 0.020 | 0.021 |
| pH 2.0 N = 2 | 0.018 | | 0.023 | |
| pH 4.5 N = 1 | 0.045 | 0.045 | 0.052 | 0.051 |
| pH 4.5 N = 2 | 0.045 | | 0.051 | |
| pH 5.5 N = 1 | 0.258 | 0.248 | 0.283 | 0.275 |
| pH 5.5 N = 2 | 0.237 | | 0.267 | |
| pH 6.8 N = 1 | 0.920 | 1.282 | 0.905 | 1.034 |
| pH 6.8 N = 2 | 1.644 | | 1.163 | |
| pH 7.4 N = 1 | 0.473 | 0.463 | 0.489 | 0.501 |
| pH 7.4 N = 2 | 0.452 | | 0.513 | |

Both visual and HPLC measurements indicate the tolcapone's solubility is pH dependent. Tolcapone is poorly soluble in acidic conditions but has improved solubility at about pH 6.8.

The lower pHs were also associated with a different microscopic physical appearance of the tolcapone crystals. Specifically, lower pH conditions promoted a form of the drug that had longer hair-like crystal structures. It was hypothesized that rearrangement into the hair-like crystals in acidic conditions provides a less soluble form such that tablets comprising tolcapone resist disintegration and erosion for drug release.

### Example 3

Tablets of the present invention were prepared with the ingredients shown in Table 3:

TABLE 3

| Component | Layer | % w/w | mg/tab |
|---|---|---|---|
| Tolcapone | Core | 27.5 | 73 |
| VivaPharm E3 (HPMC) | | 2.7 | 7.3 |
| Avicel PH-101 (MCC) | | 23.9 | 63.47 |
| Aerosil 200 (Silicon | | 0.3 | 0.73 |
| dioxide) | | | |
| Magnesium Stearate | | 0.5 | 1.46 |
| Opadry Clear | Film Coating | 1.6 | 4.38 |
| Eudragit L30 D55 | Enteric coat | 3.1 | 8.36 |
| PlasAcryl HTP20 | | 0.8 | 2.17 |
| Tolcapone | Immediate | 27.5 | 73 |
| Opadry Clear | Release | 9.1 | 24.3 |
| Opadry Brown | Top Coat | 2.9 | 7.75 |
| Total | | 100.0 | 266.0 |

The tolcapone was split between the core (50 wt %) and the immediate release layer (50 wt %).

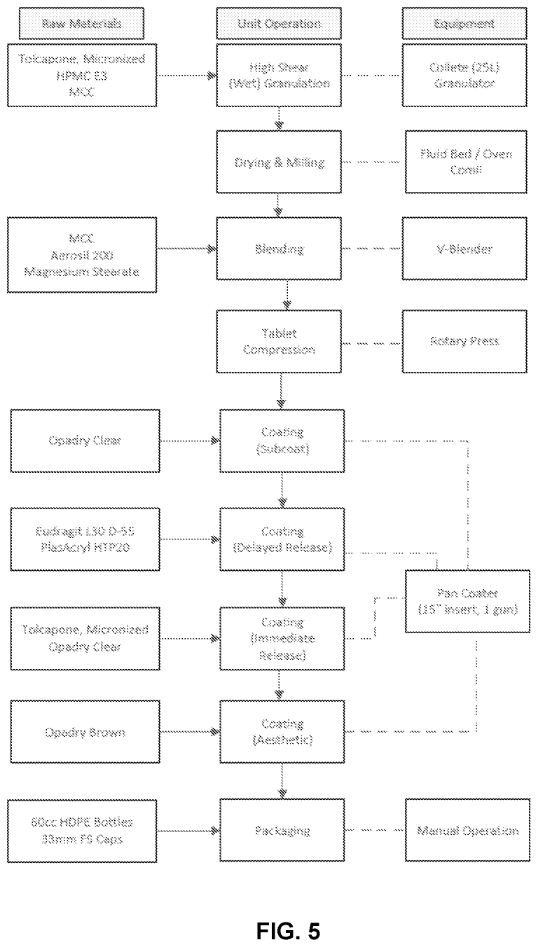

The core tablets were prepared using a wet granulation process and according to the process outlined in FIG. 5.

### Example 4

A Phase I, open-label, randomized, crossover study was conducted to evaluate the bioavailability of different tolcapone modified-release prototype formulations and to compare with Tasmar® (tolcapone immediate-release tablet) following a 300 mg dose under fasting and fed conditions in healthy subjects. The study was conducted in 2 sequential parts.

### Part 1 (Periods 1 to 6):

Part 1 was a randomized 6-period, 6-sequence, 6-treatment crossover design. Prior to entering the trial, subjects had a screening visit to establish eligibility within 28 days before study drug administration. Subjects were admitted to the clinical research facility (CRU) on Day-1 and was administered one of the 6 study Treatments on Day 1 (Period 1), Day 3 (Period 2), Day 5 (Period 3), Day 7 (Period 4), Day 9 (Period 5), and Day 11 (Period 6) under fasting conditions, in a randomized fashion. Study drug administration in each period was separated by a washout of 48 hours (±30 minutes).

### Part 2 (Periods 7 and 8):

Part 2 was a randomized, 2-period, 2-sequence, 2-treatment crossover design. Prior to entering the trial, subjects had a screening visit to establish eligibility within 28 days before study drug administration. Subjects were admitted to the CRU on Day-1 and were administered one of the 2 study Treatments on Day 1 (Period 7) and Day 3 (Period 8) under fed conditions, in a randomized fashion. Study drug administration in each period was separated by a washout of 48 hours (±30 minutes).

Part 1 and Part 2 were separated by approximately 3 weeks, the time needed to perform the interim PK analysis of Part 1. Total duration of study (Part 1 and Part 2) would be approximately 40 days. Outings were permitted during confinements. Outings were supervised at all times by the clinical site staff to ensure compliance with the protocol and was limited to the grounds surrounding the clinic.

Subjects had to be healthy, male and female, adult non-smokers, aged 18 years of age and older, with body mass index (BMI)>18.5 and <30.0 kg/m2 and body weight≥50.0 kg for males and ≥45.0 kg for females.

In each Part 1 period, the subjects received a single oral dose of one of the following treatments:

| | |
|---|---|
| Treatment A | Prototype 1 - 300 mg tolcapone modified release |
| | tablet as detailed below |
| Treatment B | Prototype 2 - 300 mg tolcapone modified release |
| | tablet with extended release core as detailed below |
| Treatment C | Prototype 3 - 300 mg tolcapone modified release |
| | tablet as detailed below |
| Treatment D | Prototype 4 - 300 mg tolcapone modified release |
| | tablet as detailed below |
| Treatment E | 100 mg tolcapone immediate release (Tasmar ®) |
| | (Valeant Pharmaceuticals North America LLC), |
| | administered as 3 × 100 mg tablets |

### Prototypes 1 & 2

TABLE 4
Compositions of Prototypes 1 & 2

| | | Prototype 1 | | Prototype 2 | |
|---|---|---|---|---|---|
| | | Immediate Release Core | | Extended Release Core | |
| | | Quantity | | Quantity | |
| | | per unit | | per unit | |
| | Function | (mg) | % w/w | (mg) | % w/w |
| Tolcapone, USP | Active substance | 299.94 | 57.70 | 299.94 | 57.70 |
| Vivapharm HPMC E3, PH. | Filler/Binder | 14.99 | 2.88 | 14.99 | 2.88 |
| Eur./USP/JP | | | | | |
| Avicel PH-101, NF/Ph. | Filler/Binder | 130.57 | 25.12 | 100.57 | 19.35 |
| Eur./JP | | | | | |
| Methocel K100 LV, | Modified | — | — | 30.00 | 5.77 |
| USP/EP/JP | Release | | | | |
| Aerosil 200, PH, | Glidant | 1.50 | 0.29 | 1.50 | 0.29 |
| NF/EP/JP | | | | | |
| Magnesium stearate, | Lubricant | 3.0 | 0.58 | 3.0 | 0.58 |
| NF/EP/JP | | | | | |
| Opadry Clear | Film coating | 59.00 | 11.35 | 59.00 | 11.35 |
| Eudragit L30D D-55, Ph. | Delayed Release | 8.60 | 1.65 | 8.60 | 1.65 |
| Eur./NF/JPE | Coat | | | | |
| PlasAcryl HTPP20 | Delayed Release | 2.20 | 0.42 | 2.20 | 0.42 |
| | Coat | | | | |
| Total | | 519.80 | 100 | 519.80 | 100 |

The first manufacturing step was high shear (wet) granulation. Three sub-batches of 68% w/w tolcapone granulation were manufactured on a GMX High Shear Granulator equipped with 4 liter bowl. The three individual sub-batches were passed through a Comil equipped with a 0.375″ sieve screen to de-lump the wet mass. The materials were dried using a FLM-1 fluid bed to a moisture level less than 3.0% (loss on drying). The dried granulations were then blended together in a 1 cubic foot V-blender. Following the blending operation, the dried granulation was passed through a Comil equipped with a 0.075″ screen.

The granulation was divided into two portions for further blending with extragranular excipients. One portion was blended in an 8 quart V-Blender with excipients to manufacture an immediate release (IR) tablet blend. The other portion was blended in the same shell with different excipients to yield an extended release (ER) tablet blend.

Compression of the IR and ER blends was performed on a rotary Piccola B/D Tablet Press with 6.5×12.5 mm caplet shaped tooling. The target weight was 300 mg to provide a 50% drug loaded tablet core. The target hardness was 10 kp in each compression run.

Each set of tablet cores were sub-coated in a CompuLab Pan Coater equipped with a 15″ pan insert. A single spray gun was used to spray the sub-coat suspension. A target weight gain of 3% was applied to the tablets.

A delayed released (DR) layer was then applied to each set of tablets using the same equipment setup. A target weight gain of 3.5% was applied to the tablets to prevent disintegration of the core tablet in the stomach environment.

An immediate release (IR) layer was then sprayed onto the tablets at a target weight gain of 62.5%. This layer provided another 150 mg of tolcapone to the formulation, bringing the total tablet strength up to 300 mg.

### Prototypes 3 & 4

Prototypes 3 & 4 are similar in size, shape and aspect. The only difference is the quantity per tablet of Hypromellose that is higher in Prototype 3. This difference provides slower dissolution profile for Prototype 3 controlled-release layer than Prototype 4 controlled-release layer.

TABLE 5
Compositions of Prototypes 3 & 4

| | | Tolcapone 300 mg | | | |
|---|---|---|---|---|---|
| | | Prototype 3 | | Prototype 4 | |
| Component and Quality | | Quantity | | Quantity | |
| Standard (and Grade, if | | per unit | | per unit | |
| applicable) | Function | (mg) | % | (mg) | % |

CONTROLLED-RELEASE LAYER

| | | | | | |
|---|---|---|---|---|---|
| Tolcapone Micronized | Active | 225 | 26.1 | 225 | 26.1 |
| USP | Pharmaceutical | | | | |
| | Ingredient | | | | |
| Croscarmellose Sodium | Disintegrant | 30 | 3.5 | 30 | 3.5 |
| Ph. Eur., USP/NF, JP | | | | | |
| Hypromellose | Controlled | 110 | 12.8 | 90 | 10.4 |
| USP, Ph. Eur., JP | Release Vehicle | | | | |
| Microcrystalline Cellulose | Filler | 65 | 7.5 | 75 | 8.7 |
| Ph. Eur., USP/NF, JP | | | | | |
| Lactose Monohydrate | Filler | 65 | 7.5 | 75 | 8.7 |
| Ph. Eur., USP/NF, JP | | | | | |
| Magnesium Stearate | Lubricant | 4 | 0.4 | 4 | 0.4 |
| Ph. Eur., USP/NF, JP | | | | | |
| Colloidal Silicon Dioxide | Glidant | 3 | 0.3 | 3 | 0.3 |
| Anhydrous | | | | | |
| Ph. Eur., USP/NF, JP | | | | | |

IMMEDIATE-RELEASE LAYER

| | | | | | |
|---|---|---|---|---|---|
| Tolcapone Micronized | Active | 75 | 8.7 | 75 | 8.7 |
| USP | Pharmaceutical | | | | |
| | Ingredient | | | | |
| Lactose Monohydrate | Filler | 205 | 23.8 | 205 | 23.8 |
| Ph. Eur., USP/NF, JP | | | | | |
| Microcrystalline Cellulose | Filler | 50 | 5.8 | 50 | 5.8 |
| Ph. Eur., USP/NF, JP | | | | | |
| Sodium Starch Glycolate | Disintegrant | 22 | 2.6 | 22 | 2.6 |
| Ph. Eur., USP/NF, JP | | | | | |
| Magnesium Stearate | Lubricant | 4 | 0.5 | 4 | 0.5 |
| Ph. Eur., USP/NF, JP | | | | | |
| Colloidal Silicon Dioxide | Glidant | 4 | 0.5 | 4 | 0.5 |
| Anhydrous | | | | | |
| Ph. Eur., USP/NF, JP | | | | | |

| | | | | |
|---|---|---|---|---|
| Total | 862 | 100 | 862 | 100 |

All ingredients, except magnesium stearate, were be blended together in a container, then magnesium stearate was added and mixed with the previously prepared blend in the same container. Afterwards, blends will be tableted using the Styl'One compression simulator.

### Blood Sampling:

PK blood samples:

A total of 16 blood samples were collected prior to drug administration and 0.5, 1.00, 1.50, 2.00, 3.00, 4.00, 5.00, 6.00, 7.00, 8.00, 9.00, 10.0, 12.0, 16.0, and 24 hours post-dose.

### PD Blood Samples:

A total of 10 blood samples were collected prior to drug administration and 1.00, 2.00, 4.00, 6.00, 8.00, 10.0, 12.0, 16.0, and 24.0 hours post-dose. The PD samples were collected and stored for future analysis.

### Criteria for Evaluation:

### Pharmacokinetic:

The following PK parameters were calculated by standard non-compartmental methods for tolcapone: AUC0-t, AUC0-inf, Residual area, Cmax, Tmax, T½ el, Kel.

### Safety:

Treatment-emergent adverse events (TEAEs), serious adverse events (SAEs), vital signs, ECG measurements (triplicate), physical examination, standard laboratory evaluations.

### Statistical Methods:

### Pharmacokinetic Analyses:

Parametric ANOVA on AUC0-t, AUC_{0-inf}, Residual area, Cₘₐₓ, T½ el, Kₑₗ; geometric confidence intervals (CI) for AUC₀₋ₜ, AUC_{0-inf}, and Cₘₐₓ; Wilcoxon non parametric test on Tmax.
Factors in the ANOVA model: Sequence, Subject within Sequence, Period and Treatment.
Ln-transformed parameters: AUC₀₋ₜ, AUC_{0-inf}, and Cₘₐₓ.

### Results:

### Pharmacokinetic:

The release profiles of Prototypes 1-4 and Tasmar® are provided in Table 6. Mean and geometric mean values for 10 subjects are presented for each type of treatment.

TABLE 6
Tolcapone Plasma Concentration vs. Nominal Time

| | Treatment A/ | | Treatment B/ | | Treatment C/ | | Treatment D/ | | Treatment E/ | |
|---|---|---|---|---|---|---|---|---|---|---|
| | Prototype 1 | | Prototype 2 | | Prototype 3 | | Prototype 4 | | Reference | |
| | (ng/mL) | | (ng/mL) | | (ng/mL) | | (ng/mL) | | (ng/mL) | |
| Time | | Geo. | | Geo. | | Geo. | | Geo. | | Geo. |
| (hour) | Mean | Mean | Mean | Mean | Mean | Mean | Mean | Mean | Mean | Mean |
| 0 | 0 | . | 0 | . | 0 | · | 0 | . | 0 | . |
| 0.5 | 6405.95 | 4399.87 | 5281.78 | 4009.74 | 2107.38 | 1406.27 | 3283.57 | 2669.93 | 4584.14 | 3440.46 |
| 1 | 7124.95 | 5280.68 | 5972.65 | 5405.19 | 4339.57 | 3575.85 | 5841.20 | 4924.87 | 6939.64 | 6022.62 |
| 1.5 | 6280.04 | 5263.29 | 5407.47 | 5098.23 | 4220.40 | 3771.47 | 6897.48 | 5623.59 | 7900.90 | 6681.47 |
| 2 | 4832.83 | 4331.78 | 4918.49 | 4666.97 | 7134.01 | 5813.99 | 5889.86 | 4908.36 | 7593.70 | 6510.99 |
| 3 | 6398.88 | 5184.85 | 3780.45 | 3526.12 | 4352.92 | 4076.22 | 4462.27 | 3778.37 | 6867.14 | 5769.35 |
| 4 | 5609.64 | 5203.38 | 2684.17 | 2527.98 | 4242.52 | 3940.00 | 4089.05 | 3733.15 | 4309.16 | 3800.08 |
| 5 | 4976.66 | 4328.38 | 2211.29 | 2061.79 | 3439.68 | 2841.16 | 2792.83 | 2724.48 | 2765.12 | 2501.50 |
| 6 | 2078.77 | 1924.29 | 1303.88 | 1165.14 | 2047.65 | 1732.11 | 1663.38 | 1564.29 | 1709.18 | 1531.36 |
| 7 | 1211.50 | 1119.54 | 784.09 | 694.34 | 1288.33 | 1122.02 | 1036.96 | 963.06 | 1112.30 | 999.06 |
| 8 | 886.46 | 816.92 | 566.22 | 484.44 | 981.12 | 830.02 | 727.82 | 685.85 | 798.48 | 690.51 |
| 9 | 803.25 | 700.40 | 475.85 | 397.69 | 804.32 | 657.09 | 599.60 | 543.16 | 606.05 | 520.13 |
| 10 | 583.31 | 522.69 | 424.04 | 352.70 | 739.61 | 554.51 | 498.38 | 424.26 | 506.06 | 428.80 |
| 12 | 286.11 | 249.76 | 219.92 | . | 346.73 | 249.55 | 221.07 | 171.14 | 232.57 | 201.74 |
| 16 | 55.85 | . | 91.34 | . | 78.16 | . | 33.19 | . | 62.56 | . |
| 24 | 0 | . | 0 | . | 0 | . | 0 | . | 0.00 | . |

The PK parameters for each Prototype are provided in Tables 7-13.

TABLE 7
Descriptive Statistics Summary of Tolcapone Pharmacokinetic
Parameters (Treatments A & B) - Part 1

| | Treatment A | | | | Treatment B | | | |
|---|---|---|---|---|---|---|---|---|
| Parameter (Unit) | N | Mean | SD | CV % | N | Mean | SD | CV % |
| AUC₀₋ₜ (h*ng/mL) | 10 | 37094.20 | 10450.11 | 28.17 | 10 | 25183.92 | 6474.79 | 25.71 |
| AUC_{0-inf} (h*ng/mL) | 10 | 37571.98 | 10514.97 | 27.99 | 9 | 25409.26 | 6926.95 | 27.26 |
| Residual Area (%) | 10 | 1.30 | 0.86 | 66.35 | 9 | 1.18 | 0.61 | 51.66 |
| Cₘₐₓ (ng/mL) | 10 | 9700.09 | 3007.94 | 31.01 | 10 | 7677.90 | 2066.52 | 26.92 |
| T_{1/2 el} (h) | 10 | 2.15 | 0.46 | 21.58 | 9 | 2.08 | 0.34 | 16.33 |
| Kₑₗ (/h) | 10 | 0.3347 | 0.0683 | 20.4073 | 9 | 0.3421 | 0.0566 | 16.5529 |
| K_{el Lower} (h) | 10 | 6.7908 | 2.4833 | 36.5682 | 9 | 3.3854 | 2.2289 | 65.8384 |
| K_{el Upper} (h) | 10 | 13.9945 | 2.1200 | 15.1486 | 9 | 14.0089 | 2.4538 | 17.5164 |
| R² Adjusted | 10 | 0.9594 | 0.0354 | 3.6846 | 10 | 0.9514 | 0.0666 | 6.9991 |
| Parameter (Unit) | N | Median | Min | Max | N | Median | Min | Max |
| Tₘₐₓ (h) | 10 | 2.999 | 0.994 | 4.988 | 10 | 1.252 | 0.499 | 3.041 |

N: Number of observations; SD: Standard Deviation; CV: Coefficient of variation; Min: Minimum; Max: Maximum.

TABLE 8
Descriptive Statistics Summary of Tolcapone Pharmacokinetic
Parameters (Treatments C & D) - Part 1

| | Treatment C | | | | Treatment D | | | |
|---|---|---|---|---|---|---|---|---|
| Parameter (Unit) | N | Mean | SD | CV % | N | Mean | SD | CV % |
| AUC₀₋ₜ (h*ng/mL) | 10 | 30064.84 | 11050.26 | 36.75 | 10 | 29199.62 | 8786.56 | 30.09 |
| AUC_{0-inf} (h*ng/mL) | 10 | 30596.95 | 11472.57 | 37.50 | 10 | 29560.86 | 8874.29 | 30.02 |
| Residual Area (%) | 10 | 1.60 | 1.26 | 78.62 | 10 | 1.24 | 0.54 | 43.29 |
| Cₘₐₓ (ng/mL) | 10 | 8545.01 | 3969.75 | 46.46 | 10 | 8637.76 | 2255.10 | 26.11 |
| T_{1/2 el} (h) | 10 | 2.12 | 0.92 | 43.37 | 10 | 1.85 | 0.34 | 18.32 |
| Kₑₗ (/h) | 10 | 0.3683 | 0.1146 | 31.1130 | 10 | 0.3859 | 0.0719 | 18.6251 |
| K_{el Lower} (h) | 10 | 8.0077 | 2.4894 | 31.0881 | 10 | 7.3956 | 2.1055 | 28.4699 |
| K_{el Upper} (h) | 10 | 14.0011 | 2.1090 | 15.0632 | 10 | 13.2026 | 1.9343 | 14.6509 |
| R² Adjusted | 10 | 0.9835 | 0.0231 | 2.3518 | 10 | 0.9733 | 0.0225 | 2.3082 |
| Parameter (Unit) | N | Median | Min | Max | N | Median | Min | Max |
| Tₘₐₓ (h) | 10 | 1.995 | 1.001 | 3.994 | 10 | 1.602 | 1.014 | 4.003 |

TABLE 9
Descriptive Statistics Summary of Tolcapone Pharmacokinetic
Parameters (Treatment E) - Part 1

| | Treatment E | | | |
|---|---|---|---|---|
| Parameter (Unit) | N | Mean | SD | CV % |
| AUC₀₋ₜ (h*ng/mL) | 10 | 34918.37 | 8359.15 | 23.94 |
| AUC_{0-inf} (h*ng/mL) | 10 | 35337.39 | 8198.20 | 23.20 |
| Residual Area (%) | 10 | 1.35 | 1.81 | 134.42 |
| Cₘₐₓ (ng/mL) | 10 | 11492.40 | 3618.40 | 31.49 |
| T_{1/2 el} (h) | 10 | 1.94 | 0.47 | 24.46 |
| Kₑₗ (/h) | 10 | 0.3720 | 0.0691 | 18.5675 |
| K_{el Lower} (h) | 10 | 5.4029 | 3.4430 | 63.7252 |
| K_{el Upper} (h) | 10 | 14.0140 | 2.1321 | 15.2138 |
| R² Adjusted | 10 | 0.9731 | 0.0254 | 2.6125 |
| Parameter (Unit) | N | Median | Min | Max |
| Tₘₐₓ (h) | 10 | 1.753 | 1.006 | 4.984 |

The bioavailability of tolcapone in the Prototype tablets was compared to Treatment E (Tasmar ®).

TABLE 10
Ratios (Treatments A/E), 90% Geometric Confidence Intervals, Intra-Subjects
CV (%), Inter-Subjects CV (%) and p-values for Tolcapone - Part 1

| | | | | 90% | | | | | | |
|---|---|---|---|---|---|---|---|---|---|---|
| | | | | Geometric | | | | | | |
| | Geometric LSM | | | C.I.² | | Intra- | Inter- | | | |
| Parameter | Treatment | Treatment | Ratio¹ | Lower | Upper | Subject | Subject | p-value | | |
| (unit) | A | E | (%) | (%) | (%) | CV (%) | CV (%) | Treatment | Period | Sequence |
| AUC₀₋ₜ | 36639.89 | 34963.24 | 104.80 | 95.04 | 115.55 | 9.74 | 30.70 | 0.3645 | 0.1973 | 0.8368 |
| (h*ng/mL) | | | | | | | | | | |
| AUC_{0-inf} | 37100.05 | 35418.46 | 104.75 | 93.61 | 117.22 | 11.23 | 29.59 | 0.4289 | 0.3223 | 0.8446 |
| (h*ng/mL) | | | | | | | | | | |
| Cₘₐₓ (ng/mL) | 9591.95 | 10789.10 | 88.90 | 73.82 | 107.07 | 18.66 | 44.90 | 0.2487 | 0.3113 | 0.7853 |

¹Calculated using least-squares means according to the formula: e^{Difference} × 100.
²90% Geometric confidence interval using In-transformed data.
LSM = Least squares mean.
Treatment A: Corino Therapeutics Inc., Prototype 1, Tolcapone 1 × 300 mg MR tablet.
Treatment E (Reference): Valeant Pharmaceuticals North America, LLC. (Tasmar ®), Tolcapone 3 × 100 mg IR tablets.
Probability (p) values are derived from Type III sums of squares.
p-value for the Sequence effect is tested using the Subject(Sequence) effect as the error term.

TABLE 11
Ratios (Treatments B/E), 90% Geometric Confidence Intervals, Intra-Subjects
CV (%), Inter-Subjects CV (%) and p-values for Tolcapone - Part 1

| | | | | 90% | | | | | | |
|---|---|---|---|---|---|---|---|---|---|---|
| | | | | Geometric | | | | | | |
| | Geometric LSM | | | C.I.² | | Intra- | Inter- | | | |
| Parameter | Treatment | Treatment | Ratio¹ | Lower | Upper | Subject | Subject | p-value | | |
| (unit) | B | E | (%) | (%) | (%) | CV (%) | CV (%) | Treatment | Period | Sequence |
| AUC_{0-inf} | 24962.48 | 34006.74 | 73.40 | 65.79 | 81.90 | 10.37 | 18.90 | 0.0038 | 0.2063 | 0.3028 |
| (h*ng/mL) | | | | | | | | | | |
| AUC₀₋ₜ | 25353.32 | 34963.24 | 72.51 | 65.37 | 80.44 | 10.95 | 22.03 | 0.0015 | 0.1353 | 0.4524 |
| (h*ng/mL) | | | | | | | | | | |
| Cₘₐₓ (ng/mL) | 7557.75 | 10789.10 | 70.05 | 51.32 | 95.62 | 33.65 | 25.41 | 0.0693 | 0.4612 | 0.7137 |

¹Calculated using least-squares means according to the formula: e^{Difference} × 100.
²90% Geometric confidence interval using In-transformed data.
LSM = Least squares mean.
Treatment B: Corino Therapeutics Inc., Prototype 2, Tolcapone 1 × 300 mg MR tablet.
Treatment E (Reference): Valeant Pharmaceuticals North America, LLC. (Tasmar ®), Tolcapone 3 × 100 mg IR tablets.
Probability (p) values are derived from Type III sums of squares.
p-value for the Sequence effect is tested using the Subject(Sequence) effect as the error term.

TABLE 12
Ratios (Treatments C/E), 90% Geometric Confidence Intervals, Intra-Subjects
CV (%), Inter-Subjects CV (%) and p-values for Tolcapone - Part 1

| | | | | 90% | | | | | | |
|---|---|---|---|---|---|---|---|---|---|---|
| | | | | Geometric | | | | | | |
| | Geometric LSM | | | C.I.² | | Intra- | Inter- | | | |
| Parameter | Treatment | Treatment | Ratio¹ | Lower | Upper | Subject | Subject | p-value | | |
| (unit) | C | E | (%) | (%) | (%) | CV (%) | CV (%) | Treatment | Period | Sequence |
| AUC₀₋ₜ | 28829.06 | 34628.00 | 83.25 | 71.14 | 97.43 | 18.25 | 32.38 | 0.0641 | 0.7571 | 0.7331 |
| (h*ng/mL) | | | | | | | | | | |
| AUC_{0-inf} | 29278.19 | 35082.04 | 83.46 | 71.15 | 97.89 | 18.51 | 31.95 | 0.0698 | 0.7847 | 0.7279 |
| (h*ng/mL) | | | | | | | | | | |
| Cₘₐₓ (ng/mL) | 8225.84 | 11242.30 | 73.17 | 50.69 | 105.61 | 44.18 | 13.26 | 0.1492 | 0.7251 | 0.5396 |

¹Calculated using least-squares means according to the formula: e^{Difference} × 100.
²90% Geometric confidence interval using In-transformed data.
Treatment C: Corino Therapeutics Inc., Prototype 3, Tolcapone 1 × 300 mg MR tablet.
Treatment E (Reference): Valeant Pharmaceuticals North America, LLC. (Tasmar ®), Tolcapone 3 × 100 mg IR tablets.
Probability (p) values are derived from Type III sums of squares.
p-value for the Sequence effect is tested using the Subject(Sequence) effect as the error term.

TABLE 13
Ratios (Treatments D/E), 90% Geometric Confidence Intervals, Intra-Subjects
CV (%), Inter-Subjects CV (%) and p-values for Tolcapone - Part 1

| | | | | 90% | | | | | | |
|---|---|---|---|---|---|---|---|---|---|---|
| | | | | Geometric | | | | | | |
| | Geometric LSM | | | C.I.² | | Intra- | Inter- | | | |
| Parameter | Treatment | Treatment | Ratio¹ | Lower | Upper | Subject | Subject | p-value | | |
| (unit) | D | E | (%) | (%) | (%) | CV (%) | CV (%) | Treatment | Period | Sequence |
| AUC₀₋ₜ | 28557.93 | 34963.24 | 81.68 | 70.87 | 94.14 | 15.03 | 27.68 | 0.0349 | 0.6071 | 0.8411 |
| (h*ng/mL) | | | | | | | | | | |
| AUC_{0-inf} | 28914.01 | 35418.46 | 81.64 | 70.81 | 94.12 | 15.07 | 26.82 | 0.0349 | 0.6268 | 0.8154 |
| (h*ng/mL) | | | | | | | | | | |
| Cₘₐₓ (ng/mL) | 8568.10 | 10789.10 | 79.41 | 65.18 | 96.76 | 21.02 | 39.53 | 0.0655 | 0.8102 | 0.9371 |

¹Calculated using least-squares means according to the formula: e^{Difference} × 100.
²90% Geometric confidence interval using In-transformed data.
Treatment D: Corino Therapeutics Inc., Prototype 4, Tolcapone 1 × 300 mg MR tablet.
Treatment E (Reference): Valeant Pharmaceuticals North America, LLC. (Tasmar ®), Tolcapone 3 × 100 mg IR tablets.
Probability (p) values are derived from Type III sums of squares.
p-value for the Sequence effect is tested using the Subject(Sequence) effect as the error term.

Treatment A showed the highest bioavailability of the Prototypes and a similar bioavailability compared to the reference product (Tasmar®). No difference in half-life was observed in the fasted condition, suggesting twice daily administration would maintain therapeutic plasma levels. Prototype 1 demonstrated a benefit of delayed absorption in the fasted state, but the half-life is not significantly longer. Therefore Prototype 1 was selected for study in Part 2 by administration under fed conditions.

TABLE 14
Treatment Description - Part 2

| Treatment | Description |
|---|---|
| Treatment G: | Prototype 1: 300 mg tolcapone MR tablet, administered |
| | as 3 × 100 mg tablet under fed conditions. |
| Treatment H: | 100 mg tolcapone IR tablets (Tasmar ®) (Valeant |
| | Pharmaceuticals North America, LLC), administered |
| | as 3 × 100 mg tablets, for a total dose of 300 mg, |
| | under fed conditions. |

For the administration of the reference product (Treatments E and H), time of dosing was set equal to the time when the first tablet was administered to the subject. If a subject was not able to swallow the 3 tablets with 240 ml of water, an additional 60 ml of water was administered and was documented. The complete dosing procedure must have been completed within 2 minutes.

A Medical Sub-Investigator was present for drug administration and until 4 hours after study drug administration to the last subject.

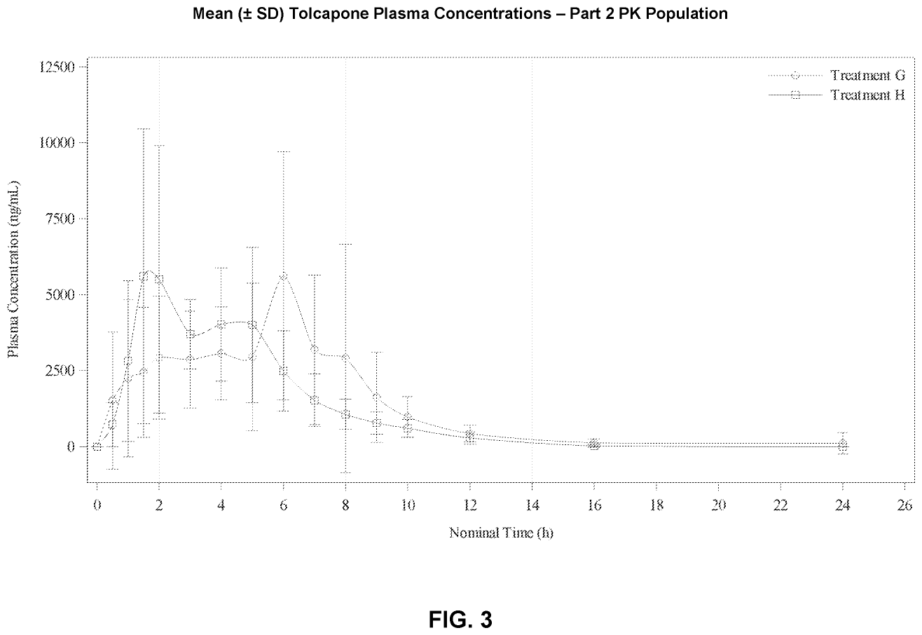

The release profile of Prototype 1 and Tasmar® under fed conditions is provided in FIG. 3. Pharmacokinetic results were as follows:

TABLE 15
Descriptive Statistics Summary of Tolcapone Pharmacokinetic Parameters - Part 2

| | Treatment G | | | | Treatment H | | | |
|---|---|---|---|---|---|---|---|---|
| Parameter (Unit) | N | Mean | SD | CV % | N | Mean | SD | CV % |
| AUC₀₋ₜ (h*ng/mL) | 10 | 30997.03 | 9978.43 | 32.19 | 9 | 27825.63 | 6791.53 | 24.41 |
| AUC_{0-inf} | 9 | 32382.39 | 10690.57 | 33.01 | 8 | 27107.34 | 6497.32 | 23.97 |
| (h*ng/mL) | | | | | | | | |
| Residual Area | 9 | 1.57 | 1.26 | 80.30 | 8 | 1.38 | 0.74 | 53.90 |
| (%) | | | | | | | | |
| Cₘₐₓ (ng/mL) | 10 | 7818.13 | 3797.78 | 48.58 | 9 | 7938.29 | 3472.30 | 43.74 |
| T_{1/2 el} (h) | 9 | 2.17 | 0.78 | 35.99 | 8 | 1.74 | 0.36 | 20.45 |
| Kₑₗ (/h) | 9 | 0.3519 | 0.1079 | 30.6629 | 8 | 0.4133 | 0.0890 | 21.5276 |
| K_{el Lower} (h) | 9 | 9.2173 | 1.2991 | 14.0941 | 8 | 7.4956 | 1.4187 | 18.9275 |
| K_{el Upper} (h) | 9 | 15.0924 | 1.7706 | 11.7317 | 8 | 13.4861 | 2.0760 | 15.3932 |
| R² Adjusted | 9 | 0.9815 | 0.0255 | 2.6017 | 9 | 0.9578 | 0.0736 | 7.6888 |
| Parameter (Unit) | N | Median | Min | Max | N | Median | Min | Max |
| Tₘₐₓ (h) | 10 | 5.996 | 2.992 | 9.991 | 9 | 1.996 | 1.492 | 5.002 |

N: Number of observations; SD: Standard Deviation; CV: Coefficient of variation; Min: Minimum; Max: Maximum.

### Treatment G (Test) Vs Treatment H (Reference)—Under Fed Conditions

The absorption of tolcapone was slightly slower when Treatment G (Prototype 1) was administered with a median peak concentration observed at 5.996 h post-dose compared to 1.996 h post dose for the Treatment H. The extent and rate (AUC and Cmax) of tolcapone absorption was similar for AUCs and approximately 15% lower for Cmax following administration of Treatment G (Prototype 1) compared to the Treatment H as expected due to differences in formulations. The mean T_{1/2 el} obtained was similar for both formulations, ranging from 2.17 hours and 1.74 hours.

The ratio of geometric means (Treatment G (Prototype 1) vs Treatment H) and 90% confidence interval (CI) were 101.68% (89.54% to 115.47), 98.72% (87.29% to 111.65%), and 85.37% (71.47% to 101.97%) for AUC0-t, AUC0-inf, and Cmax, respectively.

### Treatment G (Fed) Vs Treatment A (Fast)

The absorption of tolcapone was slightly slower when Treatment G (Fed) was administered with a median peak concentration observed at 5.996 h post-dose compared to 2.999 h post dose for the Treatment A (Fasted). The extent of tolcapone absorption (AUCs) was approximately 17% and 14% lower and rate (Cmax) was approximately 25% lower following Treatment G (Fed) compared to Treatment A (Fasted) The mean Ty, el obtained was similar for both formulations, ranging from 2.17 hours and 1.74 hours.

The ratio of geometric means (Treatment G (Fed) vs Treatment A (Fast)) and 90% confidence interval (CI) were 82.55% (66.62% to 102.29), 85.97% (68.36% to 108.11%), and 74.33% (49.90% to 110.73%) for AUC0-t, AUC0-inf, and Cmax, respectively.

ANOVA did not detect any statistically significant difference between treatments for all AUCs and Cmax.

### Treatment H-(Fed) vs Treatment E (Fast)

The absorption of tolcapone was slightly slower when Treatment H (Fed) was administered with a median peak concentration observed at 1.996 h post-dose comparatively to 1.753 h post dose for the Treatment E (Fasted). The extent of tolcapone absorption (AUCs) was 19% and 21% lower and rate (Cmax) was 28% lower following Treatment H (fed) compared to Treatment E (fasted). The mean Ty, el obtained were similar for both formulation and ranging from 1.74 hours and 1.94 hours.

The ratio of geometric means (Treatment H (Fed) vs Treatment E (Fast)) and 90% confidence interval (CI) were 81.73% (74.93% to 89.15), 78.83% (73.72% to 84.29%), and 71.53% (49.43% to 103.52%) for AUC0-t, AUC0-inf, and Cmax, respectively.

ANOVA detected a statistically significant difference between treatments for all AUCs while no statistically significant difference was detected for Cmax.

The extent and rate of absorption of Prototype 1 when administered under fed conditions were lower (from about 15% to about 25%, respectively) than when administered under fasting conditions. The extent and rate of absorption of Tasmar® was also lower (from about 19% to about 28%, respectively) when administered under fed conditions vs. fasting conditions.

Overall, the administration of single oral doses of tolcapone was safe and well tolerated in healthy subjects. Prototype 1 was most suitable when considering twice-daily administration.

### Example 5

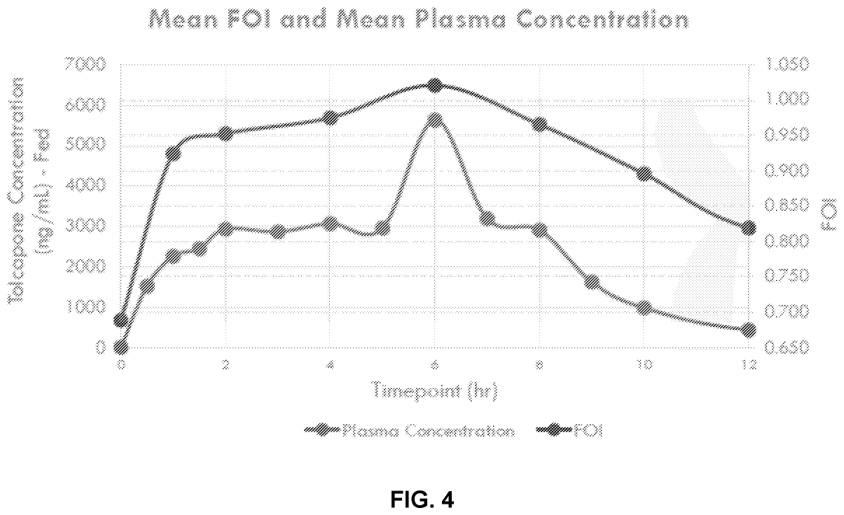

TTR stabilization was measured along with mean plasma tolcapone concentration for 12 hours following administration of Prototype 1. The fraction of initial tetramer concentration (FOI) was determined in accordance with Gamez, J., et al., “Transthyretin stabilization activity of the catechol-O-methyltranslferase inhibitor tolcapone (SOM0226) in hereditary ATTR amyloidosis patients and asymptomatic carriers: proof-of-concept study, Amyloid 2019. The results are shown in FIG. 4. Near complete TTR stabilization occurred over 12 hours with robust stabilization observed at all timepoints. Near complete stabilization is expected after repeat dosing.

1. A tablet comprising:
(i.) a tablet core comprising a first portion of tolcapone, and optionally, at least one binder, filler, glidant and/or lubricant;
(ii.) an optional film coating layer surrounding the tablet core and comprising at least one cellulosic polymer;
(iii.) an enteric coating layer surrounding the core or film coating layer and comprising acrylic acid and methacrylic acid copolymer, and optionally at least one additive;
(iv.) an immediate release layer surrounding the enteric coating layer and comprising a second portion of tolcapone and comprising at least one cellulosic polymer; and
(v.) an optional top coat surrounding the immediate release layer.

2. The tablet of claim 1, wherein the first portion of tolcapone is present in an amount from about 10 wt % to about 95 wt % by weight of the tablet core.

3. The tablet of claim 1, wherein the at least one cellulosic polymer of the optional film coating layer is present in an amount from about 0.1 wt % to about 10 wt % of the total tablet weight.

4. The tablet of claim 1, wherein the enteric coating polymer is present in an amount from about 0.5 wt % to about 10 wt % of the total tablet weight.

5. The tablet of claim 1, wherein the second portion of tolcapone is present in an amount from about 10 wt % to about 95 wt % of the total tablet weight.

6. The tablet of claim 1, wherein the at least one cellulosic polymer of the immediate release layer is present in an amount from about 5 wt % to about 20 wt % of the total tablet weight.

7. The tablet of claim 1, comprising by weight:
(i.) a tablet core comprising a first portion of tolcapone, wherein the first portion of tolcapone is present in an amount from about 10 wt % to about 95 wt %, and optionally:
a. at least one binder in an amount from about 1 wt % to about 10 wt %,
b. at least one filler in an amount from about 5 wt % to about 50 wt %,
c. at least one glidant in an amount from about 0.1 wt % to about 1 wt %, and/or at least one lubricant in an amount from about 0.1 wt % to about 1 wt %;
(ii.) an optional film coating layer surrounding the tablet core and comprising at least one cellulosic polymer, wherein the at least one cellulosic polymer is present in an amount from about 0.1 wt % to about 10 wt %;
(iii.) an enteric coating layer surrounding the core or film coating layer and comprising acrylic acid an methacrylic acid copolymer, wherein the copolymer is present in an amount from about 0.5 wt % to about 10 wt %, and optionally at least one additive in an amount from about 0.1 wt % to about 1.0 wt %;
(iv.) an immediate release layer surrounding the enteric coating layer and comprising a second portion of tolcapone and at least one cellulosic polymer, wherein the second portion of tolcapone is present in an amount from about 10 wt % to about 95 wt % and the at least one cellulosic polymer is present in an amount from about 5 wt % to about 20 wt %; and
(v.) an optional top coat surrounding the immediate release layer and comprising at least one cellulosic polymer, wherein the top coat is present in an amount from about 0.1 wt % to about 10 wt %.

8. The tablet of claim 1, comprising by weight:
(i.) a tablet core comprising a first portion of tolcapone, wherein the first portion of tolcapone is present in an amount from about 20 wt % to about 30 wt %, and optionally:
a. at least one binder in an amount from about 2 wt % to about 10 wt %,
b. at least one filler in an amount from about 20 wt % to about 30 wt %,
c. at least one glidant in an amount from about 0.1 wt % to about 0.5 wt %, and/or at least one lubricant in an amount from about 0.1 wt % to about 0.5 wt %;
(ii.) an optional film coating layer surrounding the tablet core and comprising at least one cellulosic polymer, wherein the at least one cellulosic polymer is present in an amount from about 1 wt % to about 3 wt %;
(iii.) an enteric coating layer surrounding the core or optional film coating layer and comprising acrylic acid an methacrylic acid copolymer, wherein the copolymer is present in an amount from about 1.0 wt % to about 7.0 wt %, and optionally at least one additive in an amount from about 0.1 wt % to about 1.0 wt %;
(iv.) an immediate release layer surrounding the enteric coating layer and comprising a second portion of tolcapone and at least one cellulosic polymer, wherein the second portion of tolcapone is present in an amount from about 20 wt % to about 30 wt the cellulosic polymer is present in an amount from about 5 wt % to about 15 wt %; and
(v.) an optional top coat surrounding the immediate release layer and comprising at least one cellulosic polymer, wherein the top coat is present in an amount from about 0.1 wt % to about 3.0 wt %.

9. The tablet of claim 1, wherein the tablet comprises from about 100 mg to about 600 mg tolcapone.

10. A method of treating or preventing a disease selected from transthyretin amyloidosis (ATTR), Parkinson's Disease and obsessive compulsive disorder in a patient in need thereof comprising orally administering a tablet of claim 1.

11. The method of claim 10, wherein the tablet comprises from about 100 mg to about 600 mg tolcapone.

12. The method of claim 10, wherein the tablet is administered not more than twice daily.

13. The method of claim 10, wherein the tablet is administered in a fasted or fed state.

14. The method of claim 10, wherein administration provides a blood plasma concentration (Average Cmax) of tolcapone of about 1,000 ng/ml to about 10,000 ng/mL.

15. The method of claim 10, wherein administration provides a blood plasma concentration (Average Cmin) of tolcapone of preferably no less than 200 ng/ml at 12 hours after dosing.

16. The method of claim 10, wherein administration provides a blood plasma concentration (Average Cmin) of tolcapone from about 200 ng/ml to about 800 ng/ml at 12 hours after dosing.

17. The method of claim 10, wherein, following administration to a patient, no more than 60% of the tolcapone is released within 2 hours.

18. The method of claim 10, wherein, following administration to a patient, no less than 90% of the tolcapone is released within 8 hours.

19. The method of claim 15, wherein the ATTR is selected from hereditary ATTR (hATTR), hATTR-polyneuropathy (hATTR-PN), hATTR-cardiomyopathy (hATTR-CM), ATTR-cardiomyopathy (ATTR-CM), hATTR-Leptomeningeal (hATTR-Lepto), and mixed phenotypes.

20. The method of claim 15, wherein administration provides one or more of the following compared to a reference patient treated with immediate release tolcapone: (i) an increase in the level of tetrameric TTR, (ii) a decrease the level of monomeric TTR, and/or (iii) an increase the ratio of tetrameric to monomeric TTR.
